(19) United States
(12) Patent Application Publication
(10) Pub. No.: US 2006/0058296 A1
(43) Pub. Date: Mar. 16, 2006

(54) Treatment of osteolytic lesions associated with multiple myeloma by inhibition of p38 map kinase

(75) Inventors: Linda S. Higgins, Palo Alto, CA (US); Andrew A. Protter, Palo Alto, CA (US)

Correspondence Address: MORRISON & FOERSTER LLP, 12531 HIGH BLUFF DRIVE, SUITE 100, SAN DIEGO, CA, 92130-2040, US

(73) Assignee: Scios, Inc., Fremont, CA (US)

(21) Appl. No.: 11/208,481

(22) Filed: Aug. 19, 2005

Related U.S. Application Data
(63) Continuation-in-part of application No. 11/024,170, filed on Dec. 27, 2004.
(60) Provisional application No. 60/532,439, filed on Dec. 24, 2003.
(60) Provisional application No. 60/625,636, filed on Nov. 4, 2004.

(51) Int. Cl. A61K 31/695; A61K 31/5377; A61K 31/454

(52) U.S. Cl. 514/236.5; 514/63; 514/322

(57) ABSTRACT

The present invention provides a method to treat osteolytic lesions associated with multiple myeloma by the administration of one or more p38 MAP kinase inhibitors.

### RELATED APPLICATIONS

This application is a continuation-in-part of co-pending U.S. patent application Ser. No. 11/024,170, filed Dec. 27, 2004, which claims the benefit of priority of U.S. Provisional Patent Application No. 60/532,439, filed Dec. 24, 2003 and U.S. Provisional Patent Application No. 60/625,636, filed Nov. 4, 2004, all of which are hereby incorporated by reference in their entirety.

### FIELD OF THE INVENTION

The present invention provides a method to treat osteolytic lesions caused or associated with multiple myeloma using one or more p38 MAP kinase inhibitors.

### BACKGROUND OF THE INVENTION

There are approximately 45,000 people in the United States living with multiple myeloma and an estimated 14,600 new cases of multiple myeloma are diagnosed each year. New cases of multiple myeloma thus represent twenty percent of blood cancers and one percent of all types of cancer. There is an annual incidence of multiple myeloma of approximately 4 in 100,000 in the United States. The prognosis for individuals diagnosed with multiple myeloma varies. The median survival with conventional therapies like dexamethasone, melphalan, prednisone, and bisphosphates, is about 2.5 to 3 years. Individuals treated with high dose chemotherapy and bone marrow transplant show a 5-year survival rate of greater than 50%. Although the cause of multiple myeloma is not known, risk factors for developing multiple myeloma include exposure to atomic radiation, petroleum products, pesticides, solvents, heavy metals and airborne particles.

Multiple myeloma (MM) is a neoplastic disease involving malignant plasma cells. These malignant plasma cells accumulate in bone marrow and typically produce monoclonal IgG or IgA molecules. Individuals suffering from multiple myeloma often experience anemia, osteolytic lesions, renal failure, hypercalcemia, and recurrent bacterial infections. Individuals with multiple myeloma frequently present increased monoclonal plasma cells in their bone marrow and serum or urinary monoclonal protein. For a general review of MM, see Bataille & Harousseau, JAAM (1997) 336(23): 1657-1664.

Typically, a small number of long-lived plasma cells in the bone marrow produce most of the IgG and IgA molecules found in blood serum. These plasma cells are well differentiated and do not divide. Phenotypically, plasma cells are CD38^{bright}, syndecan-1^{bright}, CD19⁺, and CD56^{weak/-}. Precursors to the plasma cells are plasmablasts that migrate from the lymph nodes after antigen stimulation to the bone marrow. Once arriving in a germinal center, these stimulated cells switch from immature IgM production to IgG or IgA. After the stimulated cells enter the bone marrow, they stop dividing and differentiate into plasma cells. Plasma cells usually undergo apoptosis and die after several weeks or months.

Myeloma cells, in contrast to the normal plasma cells, display a phenotype reminiscent of the immature plasmablast. The myeloma cells usually display CD38, syndecan-1, CD19, and CD56^{bright}, and produce low amounts of immunoglobulins. Typically, the myeloma cells are aneuploid (hypoploid, but more often hyperploid) and their chromosomes have numerous structural abnormalities. Abnormalities are frequently apparent on chromosomes 13 (13q⁻) and 14 (14q⁺). The phenotypic characteristics of cellular immaturity and the 13q⁻ and 14q⁺ abnormalities correlate with resistance to treatment and to short survival characteristics of an aggressive disease state.

The myeloma cells adhere to and activate bone marrow stromal cells (BMSC) and are long-lived. Myeloma cells are dependent on interleukin-6 (IL-6), which is produced in copious quantities by BMSC. Interleukin-6 promotes MM cell growth. Hideshima, et al., Blood (2003) 101(2):703-705. Other cytokines are thought to be involved in the growth, survival, migration and adherence of MM cells and the development of osteolytic lesions. For example, vascular endothelial growth factor (VEGF) induces MM cell migration. Id. Adherence of MM cells to bone marrow stromal (BMSC) cells up-regulates IL-6 and VEGF secretion from both MM and BMSC cells.

The p38 mitogen-activated protein kinase (MAPK), which is a member of the MAPK family of kinases that is activated by cytokines and growth factors, may play a role in the multiple myeloma disease state. The exact role of p38 in MM, however, is unknown. One recent study demonstrated that a specific inhibitor of p38 MAPK inhibited IL-6 and vascular endothelial growth factor (VEGF) secretion in bone marrow stromal cells (BMSCs) without affecting the viability of these cells (Hideshima, et al., BLOOD (2003) 101(2):703-705). TNF- alpha-induced IL-6 secretion from BMSCs was also inhibited by the specific p38 MAPK inhibitor.

### SUMMARY OF THE INVENTION

The invention is directed to methods and compounds useful in treating osteolytic lesions caused by or associated with multiple myeloma (MM). A role for p38 kinase inhibition as a treatment modality for combating multiple myeloma is discussed herein. Compounds of the invention have been found to inhibit p38 kinase, and are thus useful in treating osteolytic lesions caused by or associated with MM.

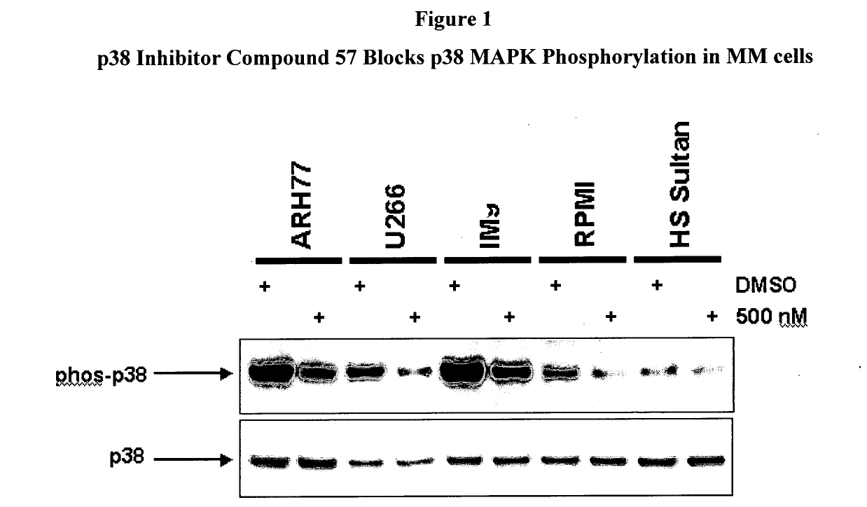

FIG. 1 shows a graphic representation of the effect of a p38 MAPK inhibitor on p38 MAPK phosphorylation in MM cells.

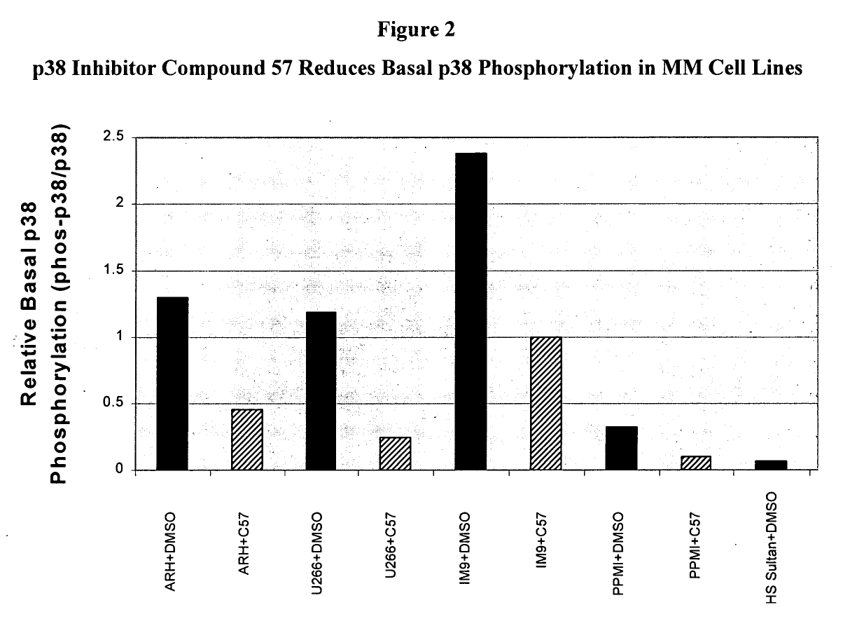

FIG. 2 shows a bar graph showing the effect of a p38 MAPK inhibitor on basal p38 phosphorylation levels in MM cell lines.

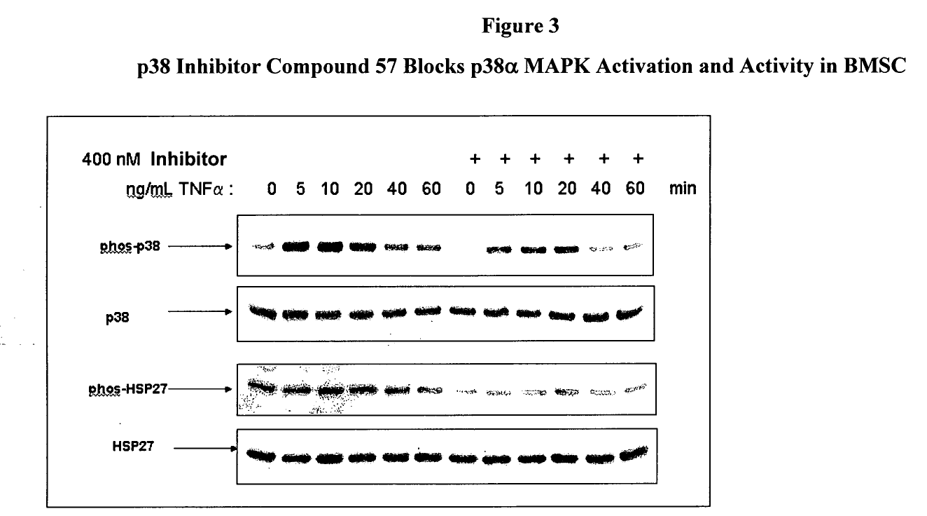

FIG. 3 shows the effect of a p38 MAPK inhibitor on p38α MAPK activation and activity in BMSC cells.

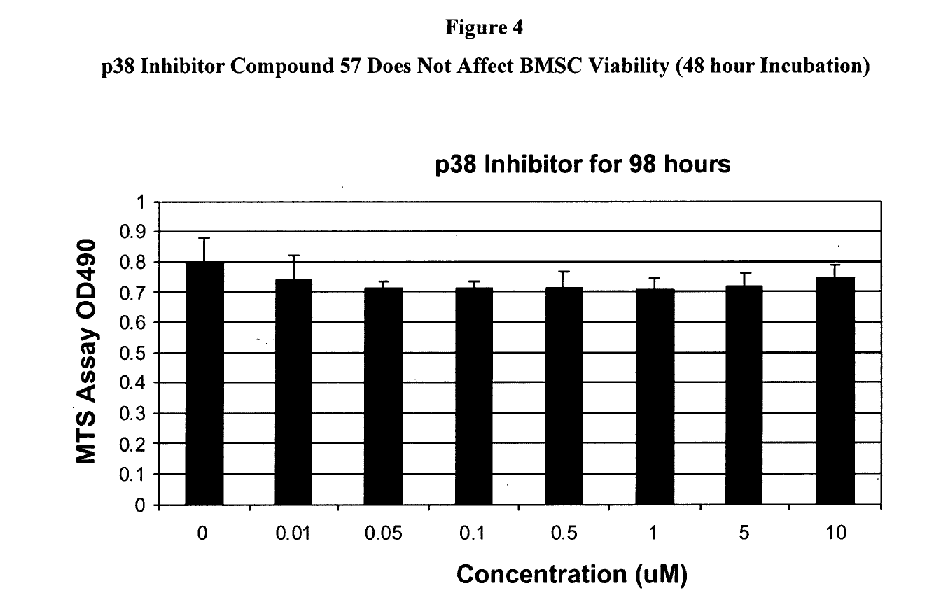

FIG. 4 shows the lack of effect of a p38 MAPK inhibitor on BMSC viability.

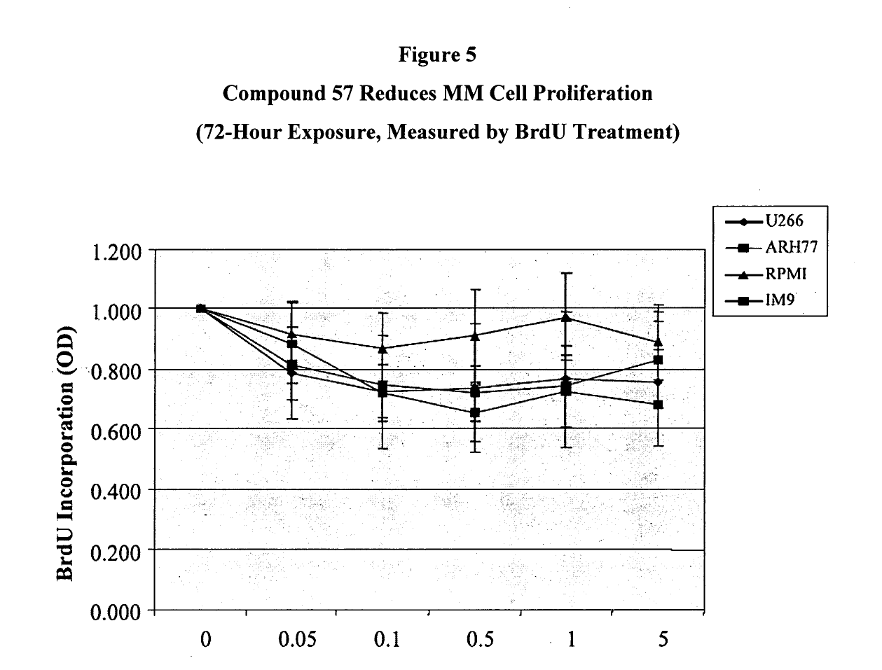

FIG. 5 shows a line graph illustrating the effect of increasing concentrations of a p38 MAPK inhibitor on MM cell proliferation.

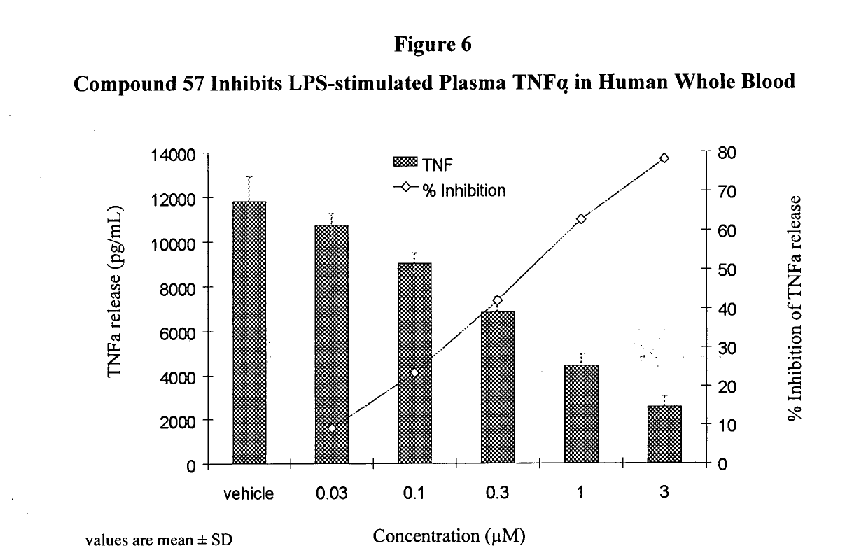

FIG. 6 shows a bar graph illustrating the effects of a p38 MAPK inhibitor on plasma TNFα levels in whole blood.

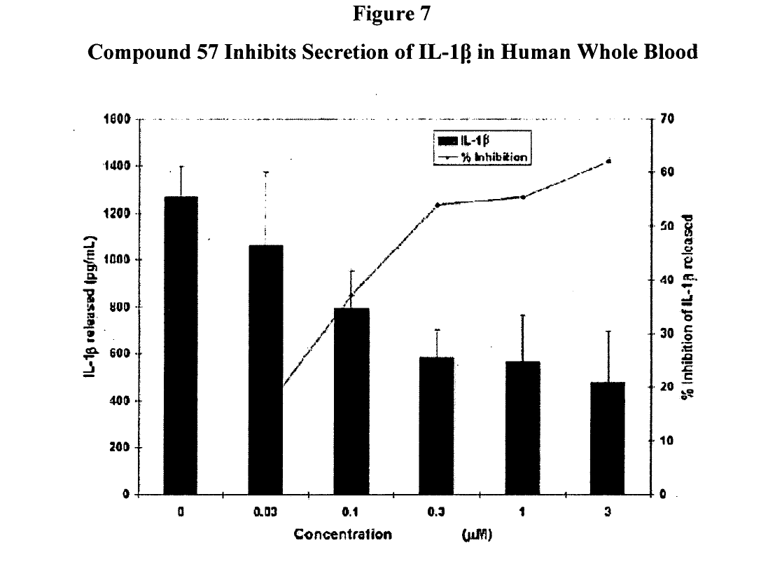

FIG. 7 shows a bar graph illustrating reduced induction levels of IL-1β in whole blood treated with a p38 MAPK inhibitor.

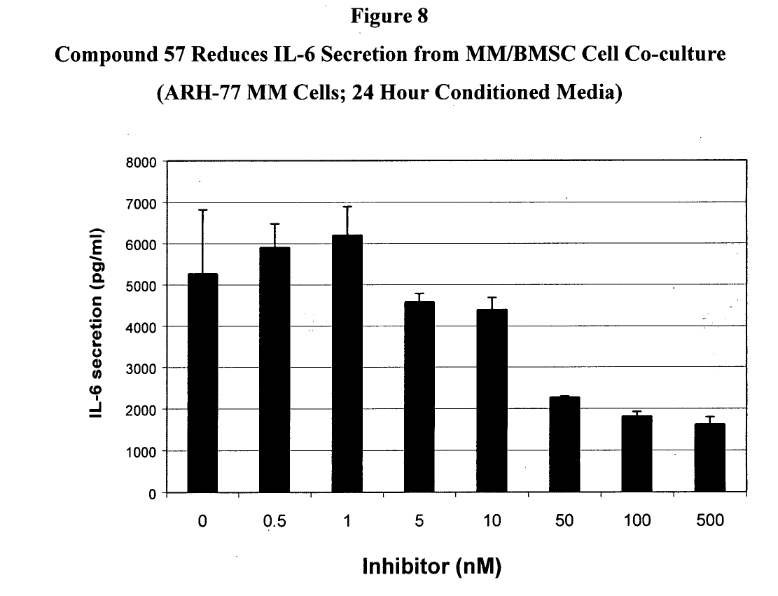

FIG. 8 shows a bar graph illustrating reduced levels of IL-6 secretion from MM/BMSC cell co-culture as a result of treatment with a p38 MAPK inhibitor.

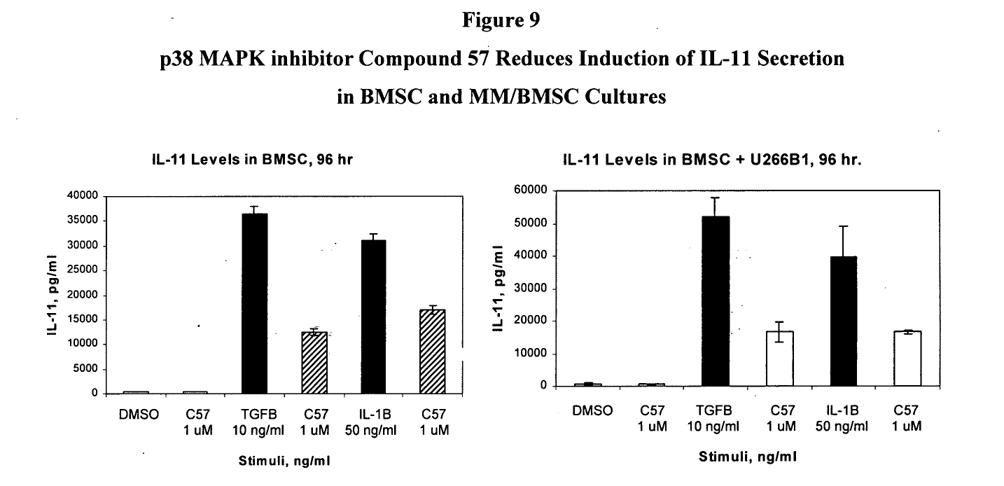

FIGS. 9A and 9B show bar graphs illustrating the reduction of IL-11 secretion in BMSC (9A) and MM/BMSC (9B) cultures as a result of treatment with a p38 MAPK inhibitor.

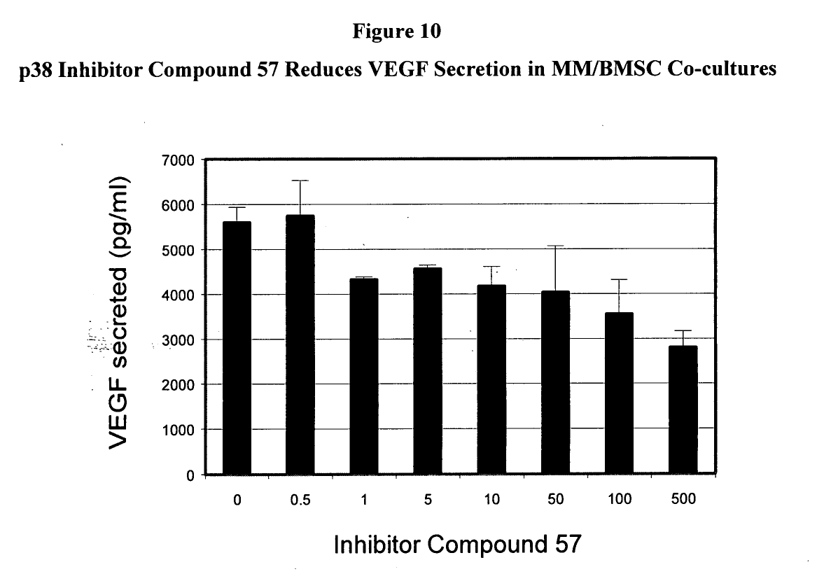

FIG. 10 shows a bar graph illustrating that treatment of MM/BMSC co-cultures with a p38 MAPK inhibitor reduces VEGF secretion.

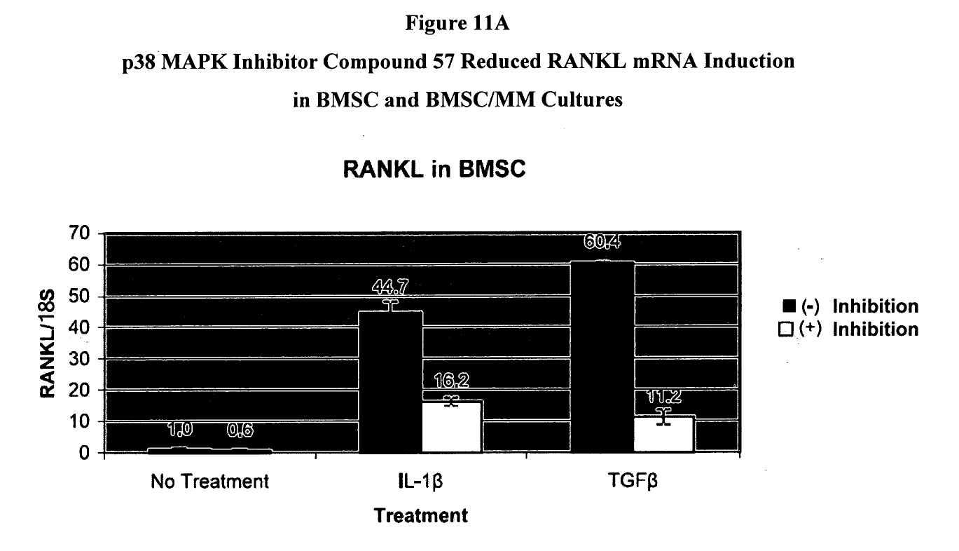

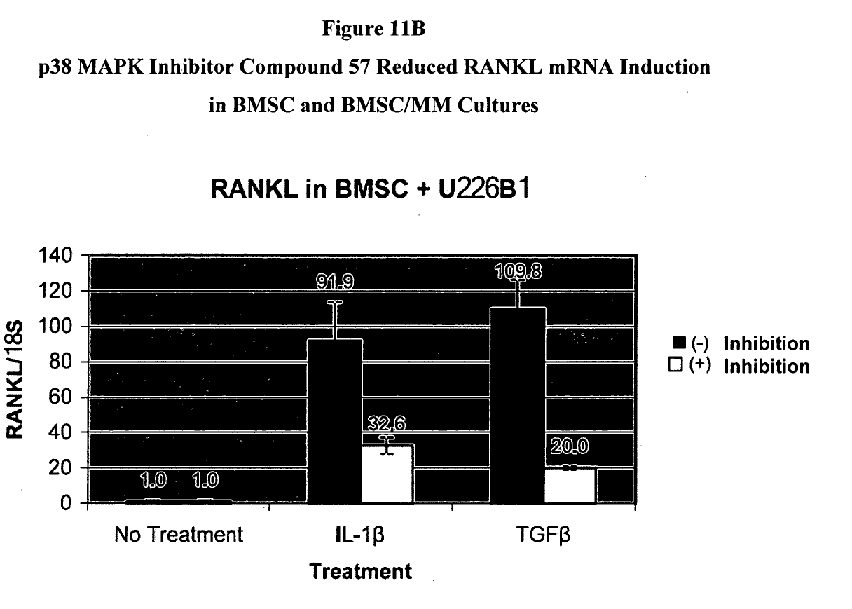

FIGS. 11A and 11B show bar graphs illustrating the reduction of RANKL mRNA induction in BMSC and BMSC/MM cultures as a result of treatment with a p38 MAPK inhibitor.

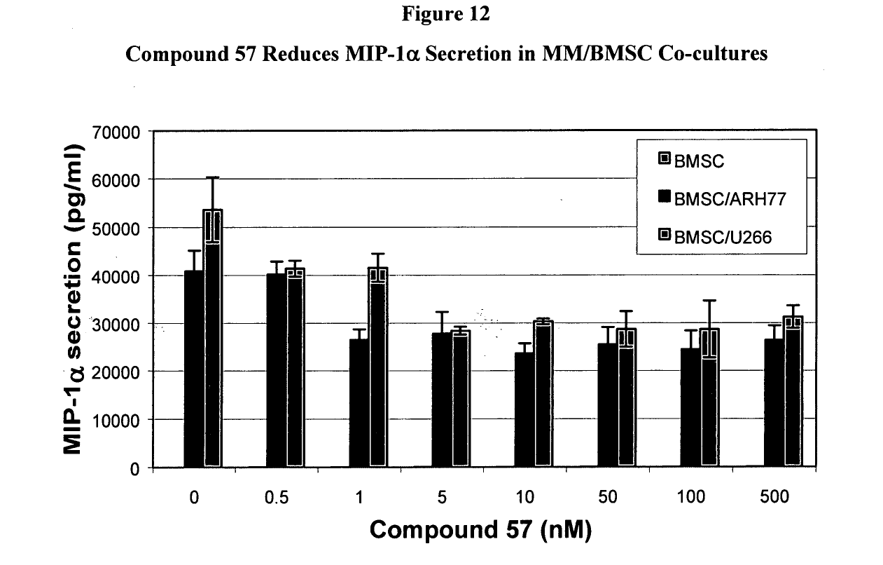

FIG. 12 is a bar graph showing that treatment with a p38 MAPK inhibitor reduces MIP-1α secretion in MM/BMSC co-cultures.

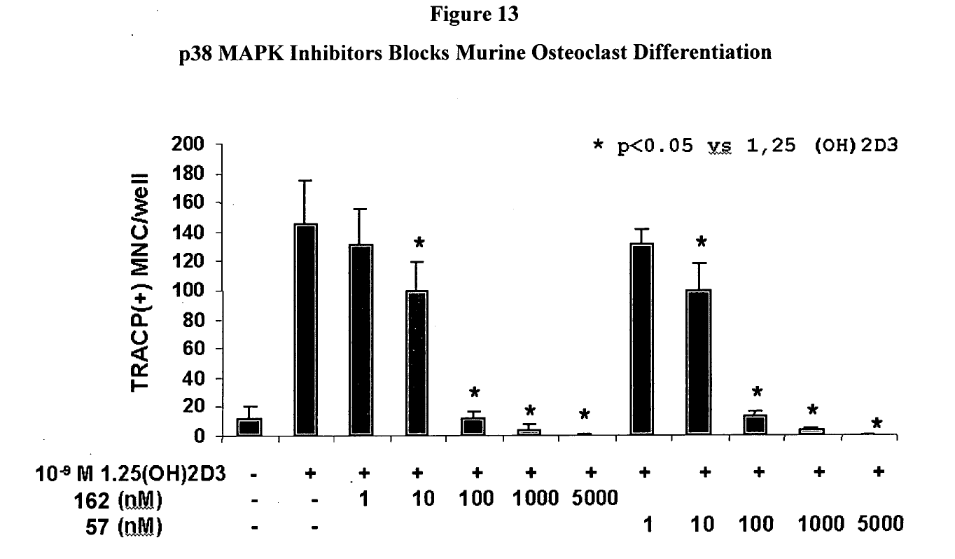

FIG. 13 is a bar graph showing how increasing doses of two different p38 MAPK inhibitors and their inhibitory effect on murine osteoclast differentiation.

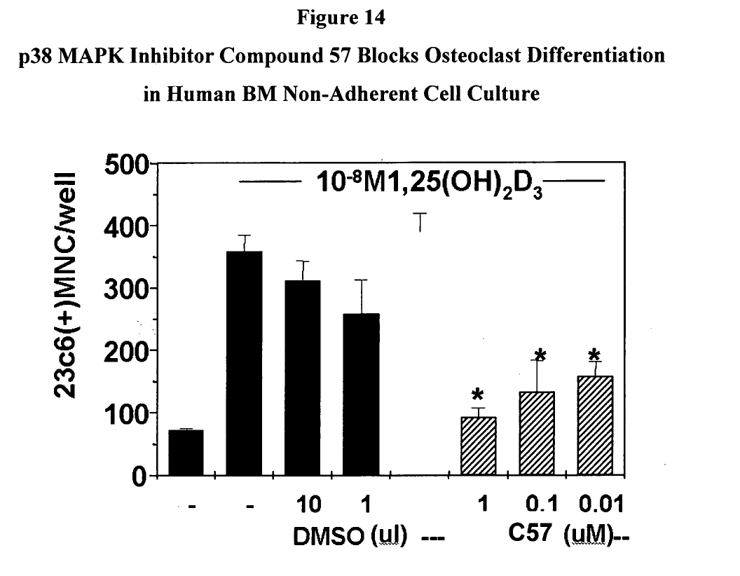

FIG. 14 is a bar graph showing the inhibitor effect that a p38 MAPK inhibitor has on osteoclast differentiation in human BM non-adherent cell culture.

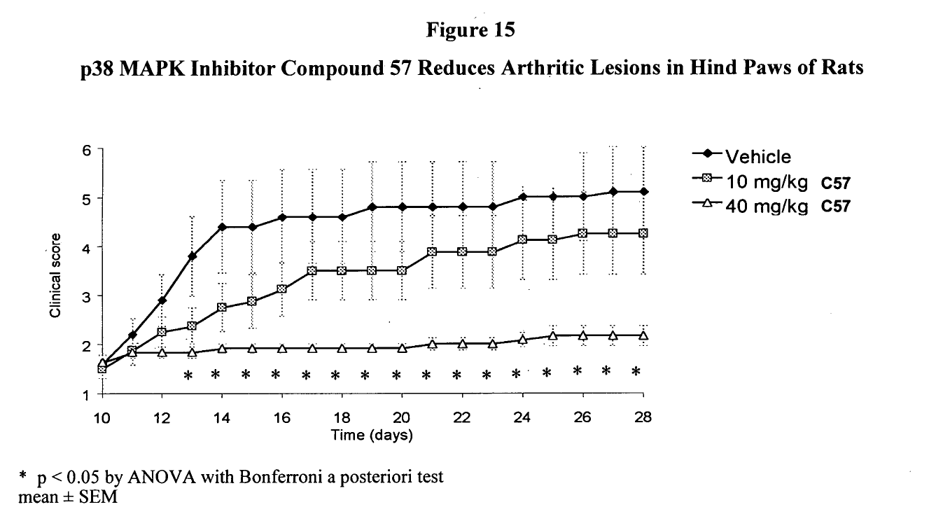

FIG. 15 is a line graph showing how administration of a p38 MAPK inhibitor reduces arthritic lesions in hind paws of rats.

### DETAILED DESCRIPTION OF THE PREFERRED EMBODIMENTS

The invention described herein relates to the use of MAP kinase inhibitors, preferably p38 MAP kinase inhibitors, either alone or in combination with other anti-osteolytic agents to prevent or reduce osteolytic lesions associated with MM. Administration of MAP kinase inhibitors generally and p38 MAP kinase inhibitors particularly, alter the microenvironment of the bone in a manner that deters osteolytic lesions and bone loss associated MM. In a preferred embodiment, small molecule antagonists of p38 MAP kinase are used to treat patients suffering from osteolytic lesions and bone loss associated with or caused by MM.

MAP Kinase Inhibitors, Cytokines and MM

Mitogen-activated protein kinases (MAPK) are activated by tyrosine and threonine phosphorylation. The p38 MAPK protein kinase family is activated primarily by cellular stresses and not mitogenic stimuli. The activation domain of p38 contains the sequence TGY, which represent the tyrosine and threonine residues required for activation (targeted by MKK3 and MKK6). The physiological role of the different p38 isoforms (which are derived from three genes as well as differential splicing) is still unclear. Among the identified targets for p38 are MAPKAPK-2 and the transcription factors, CHOP/GADD 153 (Wang and Ron, Science (1997) 272, 1347-1349), MEF2C (Han et al., Nature (1997), 386, 296-299) and ATF2.

The activation of p38 MAP kinase (phosphorylated) in MM cells and in bone marrow stromal cells (BMSC) is induced by cytokines and other inflammatory moieties present in the MM bone marrow milieu. Activation of the p38 MAP kinase may be induced even in unstimulated MM cells by tumor necrosis factor (TNF). This activation may result in the secretion of cytokines thought to be involved in the production of osteolytic lesions. Certain cytokines play a role in promoting a bone marrow microenvironment that is hospitable to the growth, survival, and migration of MM cells. Interleukin-6 (IL-6) is thought to be a primary mediator in these effects and is produced by both MM and marrow cells. Additional cytokines thought to play roles in the osteolytic pathology of MM include interleukin-1 (IL-1), interleukin-11 (IL-11), tumor necrosis factor (TNF), insulin-like growth factor-1 (IGF-1), macrophage inflammatory protein-1 (MIP-1), receptor activator of NF-kappa B ligand (RANKL), and transforming growth factor-beta (TGF-β.

Administering MAP kinase inhibitors negatively affects the bone marrow milieu in which MM cells propagate by altering cytokine expression. For example, p38 inhibitors act to reduce interleukin-6 (IL-6) production from bone marrow stromal cells (BMSCs). Production of IL-6 is thought to be important for maintaining a microenvironment that is favorable for multiple myeloma cell proliferation, that is, MM cell growth and replication. While the impact of p38 inhibitors on cytokine expression, such as IL-6 expression, is a likely mechanism by which to explain the therapeutic impact of p38 inhibitors on MM, it is not the only mechanism available to explain these positive effects. Accordingly, this mechanism is provide solely as a tool for conceptualizing the role that p38 inhibitors can play in treating osteolytic lesions caused by or associated with MM and is not intended to be limiting in any way.

MM Osteolytic Lesions and MAP Kinase Inhibitors

The constant breakdown and resynthesis of bone is mediated by bone cells that are regulated by a large number of cytokines and growth factors. Cytokine production by BMSCs and MM cells seem to play an important role in maintaining a microenvironment within the bone milieu that promotes MM cell propagation.

Multiple myeloma cells upregulate osteolytic activity as they propagate in the bone milieu, forming osteolytic lesions. Bone erosion or osteolytic lesions typically begin intramedullarly and progresses through the cortex of the bone. Radiological analysis of multiple myeloma sites is characterized by the presence of irregular osteolytic lesions of different sizes in the involved bone. The destruction of calcified bone tissue can result in hypercalcemia, which may cause confusion, weakness, lethargy, spinal cord compression and renal insufficiency in a person suffering from MM.

Administration of p38 MAP kinase inhibitors provides an effective method of preventing or reducing MM-associated osteolytic activity. For example, in one theoretical model, administering p38 kinase inhibitors prevents or reduces the osteolytic activity of MM cells by altering nuclear factor-icb activity, which facilitates osteolytic activity.

This mechanism is provided solely as a tool for conceptualizing the role that p38 inhibitors can play in treating osteolytic lesions caused by or associated with MM and is not intended to be limiting in any way.

Inhibitors of p38 MAP Kinase

As used herein, the term “inhibitor” includes, but is not limited to, any suitable molecule, compound, protein or fragment thereof, nucleic acid, formulation or substance that can regulate p38 MAP kinase activity. The inhibitor can affect a single p38 MAP kinase isoform (e.g., p38α, p38β, p38γ or p38δ), more than one isoform, or all isoforms of p38 MAP kinase. In a preferred embodiment, the inhibitor regulates the a isoform of p38 MAP kinase.

In a preferred embodiment of the disclosed invention, it is contemplated that the particular inhibitor can exhibit its regulatory effect upstream or downstream of p38 MAP kinase or on p38 MAP kinase directly. Examples of inhibitor regulated p38 MAP kinase activity include those where the inhibitor can decrease transcription and/or translation of p38 MAP kinase, can decrease or inhibit post-translational modification and/or cellular trafficking of p38 MAP kinase, or can shorten the half-life of p38 MAP kinase. The inhibitor can also reversibly or irreversibly bind p38 MAP kinase, inactivate its enzymatic activity, or otherwise interfere with its interaction with downstream substrates.

If acting on p38 MAP kinase directly, in one embodiment the inhibitor should exhibit an IC₅₀ value of about 5 μM or less, preferably about 500 nM or less, more preferably about 100 nM or less. In a related embodiment, the inhibitor should exhibit an IC₅₀ value relative to the p38α MAP kinase isoform that is about ten fold less than that observed when the same inhibitor is tested against other p38 MAP kinase isoforms in a comparable assay.

Those skilled in the art can determine whether or not a compound is useful in the disclosed invention by evaluating its p38 MAP kinase activity relative to its IC₅₀ value for p38 kinase. This evaluation can be accomplished through conventional in vitro assays. In vitro assays include assays that assess inhibition of kinase or ATPase activity of activated p38 MAP kinase. In vitro assays can also assess the ability of the inhibitor to bind to a p38 MAP kinase or to reduce or block an identified downstream effect of the activated p38 MAP kinase, e.g., cytokine secretion. IC₅₀ values are calculated using the concentration of inhibitor that causes a 50% decrease as compared to a control.

A binding assay is a fairly inexpensive and simple in vitro assay to run. As previously mentioned, binding of a molecule to p38 MAP kinase, in and of itself, can be inhibitory, due to steric, allosteric or charge-charge interactions. A binding assay can be performed in solution or on a solid phase using p38 MAP kinase or a fragment thereof as a target. By using this as an initial screen, one can evaluate libraries of compounds for potential p38 MAP kinase regulatory activity.

The target in a binding assay can be either free in solution, fixed to a support, or expressed in or on the surface of a cell. A label (e.g., radioactive, fluorescent, quenching, etc.) can be placed on the target, compound, or both to determine the presence or absence of binding. This approach can also be used to conduct a competitive binding assay to assess the inhibition of binding of a target to a natural or artificial substrate or binding partner. In any case, one can measure, either directly or indirectly, the amount of free label versus bound label to determine binding. There are many known variations and adaptations of this approach to minimize interference with binding activity and optimize signal.

For purposes of in vitro cellular assays, the compounds that represent potential inhibitors of p38 MAP kinase function can be administered to a cell in any number of ways. Preferably, the compound or composition can be added to the medium in which the cell is growing, such as tissue culture medium for cells grown in culture. The compound is provided in standard serial dilutions or in an amount determined by analogy to known modulators. Alternatively, the potential inhibitor can be encoded by a nucleic acid that is introduced into the cell wherein the cell produces the potential inhibitor itself

Alternative assays involving in vitro analysis of potential inhibitors include those where cells (e.g., HeLa) transfected with DNA coding for relevant kinases can be activated with substances such as sorbitol, IL-1, TNF, or PMA. After immunoprecipitation of cell lysates, equal aliquots of immune complexes of the kinases are pre-incubated for an adequate time with a specific concentration of the potential inhibitor followed by addition of kinase substrate buffer mix containing labeled ATP and GST-ATF2 or MBP. After incubation, kinase reactions are terminated by the addition of SDS loading buffer. Phosphorylated substrate is resolved through SDS-PAGE and visualized and quantitated in a phosphorimager. The p38 MAP kinase regulation, in terms of phosphorylation and IC₅₀ values, can be determined by quantitation. See e.g., Kumar, S. et al., Biochem. Biophys. Res. Commun. 235:533-538 (1997). Similar techniques can be used to evaluate the effects of potential inhibitors on other MAP kinases.

Other in vitro assays can assess the production of TNF-a as a correlation to p38 MAP kinase activity. One such example is a Human Whole Blood Assay. In this assay, venous blood is collected from, e.g., healthy male volunteers into a heparinized syringe and is used within 2 hours of collection. Test compounds are dissolved in 100% DMSO and 1 μl aliquots of drug concentrations ranging from 0 to 1 mM are dispensed into quadruplicate wells of a 24-well microtiter plate (Nunclon Delta SI, Applied Scientific Co., San Francisco, Calif.). Whole blood is added at a volume of 1 ml/well and the mixture is incubated for 15 minutes with constant shaking (Titer Plate Shaker, Lab-Line Instruments, Inc., Melrose Park, Ill.) at a humidified atmosphere of 5% CO₂ at 37° C. Whole blood is cultured either undiluted or at a final dilution of 1:10 with RPMI 1640 (Gibco 31800+NaHCO₃, Life Technologies, Rockville, Md. and Scios, Inc., Sunnyvale, Calif.). At the end of the incubation period, 10 μl of LPS (E. coli 0111:B4, Sigma Chemical Co., St. Louis, Mo.) is added to each well to a final concentration of 1 or 0.1 μg/ml for undiluted or 1:10 diluted whole blood, respectively. The incubation is continued for an additional 2 hours. The reaction is stopped by placing the microtiter plates in an ice bath, and plasma or cell-free supernates are collected by centrifugation at 3000 rpm for 10 minutes at 4° C. The plasma samples are stored at −80° C. until assayed for TNF-α levels by ELISA, following the directions supplied by Quantikine Human TNF-α assay kit (R&D Systems, Minneapolis, Minn.). IC₅₀ values are calculated using the concentration of inhibitor that causes a 50% decrease as compared to a control.

A similar assay is an Enriched Mononuclear Cell Assay. The enriched mononuclear cell assay begins with cryopreserved Human Peripheral Blood Mononuclear Cells (HPBMCs) (Clonetics Corp.) that are rinsed and resuspended in a warm mixture of cell growth media. The resuspended cells are then counted and seeded at 1×10⁶ cells/well in a 24-well microtitre plate. The plates are then placed in an incubator for an hour to allow the cells to settle in each well. After the cells have settled, the media is aspirated and new media containing 100 ng/ml of the cytokine stimulatory factor Lipopolysaccharide (LPS) and a test chemical compound is added to each well of the microtiter plate. Thus, each well contains HPBMCs, LPS and a test chemical compound. The cells are then incubated for 2 hours, and the amount of the cytokine Tumor Necrosis Factor Alpha (TNF-α) is measured using an Enzyme Linked Immunoassay (ELISA). One such ELISA for detecting the levels of TNF-α is commercially available from R&D Systems. The amount of TNF-a production by the HPBMCs in each well is then compared to a control well to determine whether the chemical compound acts as an inhibitor of cytokine production.

While IC₅₀ values are an initial indicia for identifying compounds that are useful for the invention, it is contemplated that one skilled in the art would further consider additional and conventional pharmaceutical considerations including but not limited to bioavailability, pK values, routes of delivery, solubility, and the like.

Exemplary Inhibitors

Preferred examples of the compounds of the invention are of the formula: 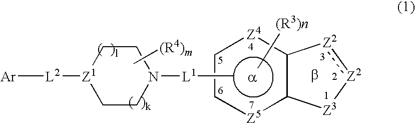
and the pharmaceutically acceptable salts thereof, or a pharmaceutical composition thereof, wherein
[symbol] represents a single or double bond;
one Z² is CA or CR 8A and the other is CR¹, CR¹₂, NR⁶ or N wherein each R¹, R⁶ and R⁸ is independently hydrogen or noninterfering substituent;
A is —W₁—COXⱼY wherein Y is COR² or an isostere thereof and R² is hydrogen or a noninterfering substituent, each of W and X is a spacer of 2-6 Å, and each of i and j is independently 0 or 1;
Z³ is NR⁷ or O;
each of Z⁴ and Z⁵ is independently N or CR¹ wherein R¹ is as defined above and wherein at least one of Z⁴ and Z⁵ is N;
each R³ is independently a noninterfering substituent;
n is 0-3;
each of L¹ and L² is a linker;
each R⁴ is independently a noninterfering substituent;
m is 0-4;
Z¹ is CR⁵ or N wherein R⁵ is hydrogen or a noninterfering substituent;
each of l and k is an integer from 0-2 wherein the sum of l and k is 0-3;

Ar is an aryl group substituted with 0-5 noninterfering substituents, wherein two noninterfering substituents can form a fused ring.

Preferred embodiments of compounds useful in the invention are derivatives of indole-type compounds containing a mandatory substituent, A, at a position corresponding to the 2- or 3-position of indole. In general, an indole-type nucleus is preferred, although alternatives within the scope of the invention are also illustrated below. Additionally, PCT publication WO00/71535, published 7 Dec. 2000, discloses indole derived compounds that are specific inhibitors of p38 kinase. The disclosure of this document is incorporated herein by reference.

U.S. Provisional Patent Application No. 60/417,599 filed 9 Oct. 2002 and U.S. patent application Ser. No. 10/683,656, filed Oct. 9, 2003, disclose azaindole derivatives that are useful in treating conditions that are characterized by enhanced p38 activity and are therefore useful for purposes of this invention. The disclosure of these documents is incorporated herein by reference.

As used herein, a “noninterfering substituent” is a substituent which either leaves the ability of the compound of formula (1) to inhibit p38-a activity qualitatively intact or enhances the activity of the inhibitor. Thus, the substituent may alter the degree of inhibition of p38. However, as long as the compound of formula (1) retains the ability to inhibit p38 activity, the substituent will be classified as “noninterfering.” As mentioned above, a number of assays for determining the ability of any compound to inhibit p38 activity are available in the art. A whole blood assay for this evaluation is illustrated below: the gene for p38 has been cloned and the protein can be prepared recombinantly and its activity assessed, including an assessment of the ability of an arbitrarily chosen compound to interfere with this activity. The essential features of the molecule are tightly defined. The positions which are occupied by “noninterfering substituents” can be substituted by conventional organic moieties as is understood in the art. It is irrelevant to the present invention to test the outer limits of such substitutions.

Regarding the compounds of formula (1), L¹ and L² are described herein as linkers. The nature of such linkers is typically less important that the distance they impart between the portions of the molecule. Typical linkers include alkylene, i.e. (CH₂)ₙ—R; alkenylene—i.e., an alkylene moiety which contains a double bond, including a double bond at one terminus. Other suitable linkers include, for example, substituted alkylenes or alkenylenes, carbonyl moieties, and the like.

As used herein, “hydrocarbyl residue” refers to a residue which contains only carbon and hydrogen. The residue may be aliphatic or aromatic, straight-chain, cyclic, branched, saturated or unsaturated. The hydrocarbyl residue, when so stated however, may contain heteroatoms over and above the carbon and hydrogen members of the substituent residue. Thus, when specifically noted as containing such heteroatoms, the hydrocarbyl residue may also contain carbonyl groups, amino groups, hydroxyl groups and the like, or contain heteroatoms within the “backbone” of the hydrocarbyl residue.

As used herein, “inorganic residue” refers to a residue that does not contain carbon. Examples include, but are not limited to, halo, hydroxy, NO₂ or NH₂.

As used herein, the term “alkyl,” “alkenyl” and “alkynyl” include straight- and branched-chain and cyclic monovalent substituents. Examples include methyl, ethyl, isobutyl, cyclohexyl, cyclopentylethyl, 2-propenyl, 3-butynyl, and the like. Typically, the alkyl, alkenyl and alkynyl substituents contain 1-10C (alkyl) or 2-10C (alkenyl or alkynyl). Preferably they contain 1-6C (alkyl) or 2-6C (alkenyl or alkynyl). Heteroalkyl, heteroalkenyl and heteroalkynyl are similarly defined but may contain 1-2 O, S or N heteroatoms or combinations thereof within the backbone residue.

As used herein, “acyl” encompasses the definitions of alkyl, alkenyl, alkynyl and the related hetero-forms which are coupled to an additional residue through a carbonyl group.

“Aromatic” moiety refers to a monocyclic or fused bicyclic moiety such as phenyl or naphthyl; “heteroaromatic” also refers to monocyclic or fused bicyclic ring systems containing one or more heteroatoms selected from O, S and N. The inclusion of a heteroatom permits inclusion of 5-membered rings as well as 6-membered rings. Thus, typical aromatic systems include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, imidazolyl and the like. Any monocyclic or fused ring bicyclic system which has the characteristics of aromaticity in terms of electron distribution throughout the ring system is included in this definition. Typically, the ring systems contain 5-12 ring member atoms.

Similarly, “arylalkyl” and “heteroalkyl” refer to aromatic and heteroaromatic systems which are coupled to another residue through a carbon chain, including substituted or unsubstituted, saturated or unsaturated, carbon chains, typically of 1-6C. These carbon chains may also include a carbonyl group, thus making them able to provide substituents as an acyl moiety.

When the compounds of Formula 1 contain one or more chiral centers, the invention includes optically pure forms as well as mixtures of stereoisomers or enantiomers.

With respect to the portion of the compound of formula (1) between the atom of Ar bound to L² and ring α, L¹ and L² are linkers which space the substituent Ar from ring α at a distance of 4.5-24 Å, preferably 6-20 Å, more preferably 7.5-10 Å. In a preferred embodiment, the distance of substituent Ar from ring is less than 24 Å. The distance is measured from the center of the a ring to the atom of Ar to which the linker L² is attached. Typical, but nonlimiting, embodiments of L¹ and L² are CO and isosteres thereof, or optionally substituted isosteres, or longer chain forms. L², in particular, may be alkylene or alkenylene optionally substituted with noninterfering substituents or L¹ or L² may be or may include a heteroatom such as N, S or O. Such substituents include, but are limited to, a moiety selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOR, SO₃R, CONR₂, SO₂NR₂, NRSO₂NR₂, CN, CF₃, R₃Si, and NO₂, wherein each R is independently H, alkyl, alkenyl or aryl or heteroforms thereof, and wherein two substituents on L² can be joined to form a non-aromatic saturated or unsaturated ring that includes 0-3 heteroatoms which are O, S and/or N and which contains 3 to 8 members or said two substituents can be joined to form a carbonyl moiety or an oxime, oximeether, oximeester or ketal of said carbonyl moiety.

Isosteres of CO and CH₂, include SO, SO₂, or CHOH. CO and CH₂ are preferred.

Thus, L² is substituted with 0-2 substituents. Where appropriate, two optional substituents on L² can be joined to form a non-aromatic saturated or unsaturated hydrocarbyl ring that includes 0-3 heteroatoms such as O, S and/or N and which contains 3 to 8 members. Two optional substituents on L² can be joined to form a carbonyl moiety which can be subsequently converted to an oxime, an oximeether, an oximeester, or a ketal.

Ar is aryl, heteroaryl, including 6-5 fused heteroaryl, cycloaliphatic or cycloheteroaliphatic that can be optionally substituted. Ar is preferably optionally substituted phenyl.

Each substituent on Ar is independently a hydrocarbyl residue (1-20C) containing 0-5 heteroatoms selected from O, S and N, or is an inorganic residue. Preferred substituents include those selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOR, SO₃R, CONR₂, SO₂NR₂, NRSO₂NR₂, CN, CF₃, R₃Si, and NO₂, wherein each R is independently H, alkyl, alkenyl or aryl or heteroforms thereof, and wherein two of said optional substituents on adjacent positions can be joined to form a fused, optionally substituted aromatic or nonaromatic, saturated or unsaturated ring which contains 3-8 members. More preferred substituents include halo, alkyl (1-4C) and more preferably, fluoro, chloro and methyl. These substituents may occupy all available positions of the aryl ring of Ar, preferably 1-2 positions, most preferably one position. These substituents may be optionally substituted with substituents similar to those listed. Of course some substituents, such as halo, are not further substituted, as known to one skilled in the art.

Two substituents on Ar can be joined to form a fused, optionally substituted aromatic or nonaromatic, saturated or unsaturated ring which contains 3-8 members.

Regarding formula (1), between L¹ and L² is a piperidine-type moiety of the following formula: 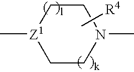
Z¹ is CR⁵ or N wherein R⁵ is H or a noninterfering substituent. Each of l and k is an integer from 0-2 wherein the sum of l and k is 0-3. The noninterfering substituents R⁵ include, without limitation, halo, alkyl, alkoxy, aryl, arylalkyl, aryloxy, heteroaryl, acyl, carboxy, or hydroxy. Preferably, R⁵ is H, alkyl, OR, NR₂, SR or halo, where R is H or alkyl. Additionally, R⁵ can be joined with an R⁴ substituent to form an optionally substituted non-aromatic saturated or unsaturated hydrocarbyl ring which contains 3-8 members and 0-3 heteroatoms such as O, N and/or S. Preferred embodiments include compounds wherein Z¹ is CH or N, and those wherein both l and k are 1.

R⁴ represents a noninterfering substituent such as a hydrocarbyl residue (1-20C) containing 0-5 heteroatoms selected from O, S and N. Preferably R⁴ is alkyl, alkoxy, aryl, arylalkyl, aryloxy, heteroalkyl, heteroaryl, heteroarylalkyl, RCO, ═O, acyl, halo, CN, OR, NRCOR, NR, wherein R is H, alkyl (preferably 1-4C), aryl, or hetero forms thereof. Each appropriate substituent is itself unsubstituted or substituted with 1-3 substituents. The substituents are preferably independently selected from a group that includes alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOR, SO₃R, CONR₂, SO₂NR₂, NRSO₂NR₂, CN, CF₃, R₃Si, and NO₂, wherein each R is independently H, alkyl, alkenyl or aryl or heteroforms thereof and two of R⁴ on adjacent positions can be joined to form a fused, optionally substituted aromatic or nonaromatic, saturated or unsaturated ring which contains 3-8 members, or R⁴ is ═O or an oxime, oximeether, oximeester or ketal thereof. R⁴ may occur m times on the ring; m is an integer of 0-4. Preferred embodiments of R⁴ comprise alkyl (1-4C) especially two alkyl substituents and carbonyl. Most preferably R⁴ comprises two methyl groups at positions 2 and 5 or 3 and 6 of a piperidinyl or piperazinyl ring or =0 preferably at the 5-position of the ring. The substituted forms may be chiral and an isolated enantiomer may be preferred.

R³ also represents a noninterfering substituent. Such substituents include hydrocarbyl residues (1-6C) containing 0-2 heteroatoms selected from O, S and/or N and inorganic residues. n is an integer of 0-3, preferably 0 or 1. Preferably, the substituents represented by R³ are independently halo, alkyl, heteroalkyl, OCOR, OR, NRCOR, SR, or NR₂, wherein R is H, alkyl, aryl, or heteroforms thereof. More preferably R³ substituents are selected from alkyl, alkoxy or halo, and most preferably methoxy, methyl, and chloro. Most preferably, n is 0 and the a ring is unsubstituted, except for L¹ or n is 1 and R³ is halo or methoxy.

In the ring labeled β, Z³ may be NR⁷ or O—i.e., the compounds may be related to indole or benzofuran. If C³ is NR⁷, preferred embodiments of R⁷ include H or optionally substituted alkyl, alkenyl, alkynyl, aryl, arylalkyl, acyl, aroyl, heteroaryl, heteroalkyl, heteroalkenyl, heteroalkynyl, heteroalkylaryl, or is SOR, SO₂R, RCO, COOR, alkyl-COR, SO₃R, CONR₂, SO₂NR₂, CN, CF₃, NR₂, OR, alkyl-SR, alkyl-SOR, alkyl-SO₂R, alkyl-OCOR, alkyl-COOR, alkyl-CN, alkyl-CONR₂, or R₃Si, wherein each R is independently H, alkyl, alkenyl or aryl or heteroforms thereof. More preferably, R⁷ is hydrogen or is alkyl (1-4C), preferably methyl or is acyl (1-4C), or is COOR wherein R is H, alkyl, alkenyl of aryl or hetero forms thereof. R⁷ is also preferably a substituted alkyl wherein the preferred substituents are form ether linkages or contain sulfinic or sulfonic acid moieties. Other preferred substituents include sulfhydryl substituted alkyl substituents. Still other preferred substituents include CONR₂ wherein R is defined as above.

It is preferred that the indicated dotted line represents a double bond; however, compounds which contain a saturated β ring are also included within the scope of the invention.

Preferably, the mandatory substituent CA or CR⁸A is in the 3-position; regardless of which position this substituent occupies, the other position is CR¹, CR¹₂, NR⁶ or N. CR¹ is preferred. Preferred embodiments of R¹ include hydrogen, alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOR, SO₃R, CONR₂, SO₂NR₂, NRSO₂NR₂, CN, CF₃, R₃Si, and NO₂, wherein each R is independently H, alkyl, alkenyl or aryl or heteroforms thereof and two of R¹ can be joined to form a fused, optionally substituted aromatic or nonaromatic, saturated or unsaturated ring which contains 3-8 members. Most preferably, R¹ is H, alkyl, such as methyl, most preferably, the ring labeled a contains a double bond and CR¹ is CH or C-alkyl. Other preferable forms of R¹ include H, alkyl, acyl, aryl, arylalkyl, heteroalkyl, heteroaryl, halo, OR, NR₂, SR, NRCOR, alkyl-OOR, RCO, COOR, and CN, wherein each R is independently H, alkyl, or aryl or heteroforms thereof.

While the position not occupied by CA is preferred to include CR¹, the position can also be N or NR⁶. While NR⁶ is less preferred (as in that case the ring labeled β would be saturated), if NR is present, preferred embodiments of R⁶ include H, or alkyl, alkenyl, alkynyl, aryl, arylalkyl, acyl, aroyl, heteroaryl, heteroalkyl, heteroalkenyl, heteroalkynyl, heteroalkylaryl, or is SOR, SO₂R, RCO, COOR, alkyl-COR, SO₃R, CONR₂, SO₂NR₂, CN, CF₃, or R₃Si wherein each R is independently H, alkyl, alkenyl or aryl or heteroforms thereof.

Preferably, CR⁸A or CA occupy position 3- and preferably Z² in that position is CA. However, if the β ring is saturated and R⁸ is present, preferred embodiments for R⁸ include H, halo, alkyl, alkenyl and the like. Preferably R⁸ is a relatively small substituent corresponding, for example, to H or lower alkyl 1-4C.

A is —Wᵢ—COXⱼY wherein Y is COR² or an isostere thereof and R² is a noninterfering substituent. Each of W and X is a spacer and may be, for example, optionally substituted alkyl, alkenyl, or alkynyl, each of i and j is 0 or 1. Preferably, W and X are unsubstituted. Preferably, j is 0 so that the two carbonyl groups are adjacent to each other. Preferably, also, i is 0 so that the proximal CO is adjacent the ring. However, compounds wherein the proximal CO is spaced from the ring can readily be prepared by selective reduction of an initially glyoxal substituted β ring. In the most preferred embodiments of the invention, the α/β ring system is an indole containing CA in position 3- and wherein A is COCR².

The noninterfering substituent represented by R², when R² is other than H, is a hydrocarbyl residue (1-20C) containing 0-5 heteroatoms selected from O, S and/or N or is an inorganic residue. Preferred are embodiments wherein R² is H, or is straight or branched chain alkyl, alkenyl, alkynyl, aryl, arylalkyl, heteroalkyl, heteroaryl, or heteroarylalkyl, each optionally substituted with halo, alkyl, heteroalkyl, SR, OR, NR₂, OCOR, NRCOR, NRCONR₂, NRSO₂R, NRSO₂NR₂, OCONR₂, CN, COOR, CONR₂, COR, or R₃Si wherein each R is independently H, alkyl, alkenyl or aryl or the heteroatom-containing forms thereof, or wherein R² is OR, NR₂, SR, NRCONR₂, OCONR₂, or NRSO₂NR₂, wherein each R is independently H, alkyl, alkenyl or aryl or the heteroatom-containing forms thereof, and wherein two R attached to the same atom may form a 3-8 member ring and wherein said ring may further be substituted by alkyl, alkenyl, alkynyl, aryl, arylalkyl, heteroalkyl, heteroaryl, heteroarylalkyl, each optionally substituted with halo, SR, OR, NR₂, OCOR, NRCOR, NRCONR₂, NRSO₂R, NRSO₂NR₂, OCONR₂, or R₃Si wherein each R is independently H, alkyl, alkenyl or aryl or the heteroatom-containing forms thereof wherein two R attached to the same atom may form a 3-8 member ring, optionally substituted as above defined.

Other preferred embodiments of R² are H, heteroarylalkyl, —NR₂, heteroaryl, —COOR, —NHRNR₂, heteroaryl-COOR, heteroaryloxy, —OR, heteroaryl-NR₂, —NROR and alkyl. Most preferably R² is isopropyl piperazinyl, methyl piperazinyl, dimethylamine, piperazinyl, isobutyl carboxylate, oxycarbonylethyl, morpholinyl, aminoethyldimethylamine, isobutyl carboxylate piperazinyl, oxypiperazinyl, ethylcarboxylate piperazinyl, methoxy, ethoxy, hydroxy, methyl, amine, aminoethyl pyrrolidinyl, aminopropanediol, piperidinyl, pyrrolidinyl-piperidinyl, or methyl piperidinyl.

Isosteres of COR² as represented by Y are defined as follows.

The isosteres have varying lipophilicity and may contribute to enhanced metabolic stability. Thus, Y, as shown, may be replaced by the isosteres in Table 1.

TABLE 1

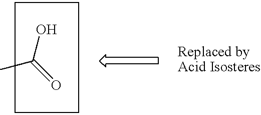

Acid Isosteres

| Names of Groups | Chemical Structures | Substitution Groups (SG) |
|---|---|---|
| tetrazole | 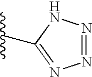 | n/a |
| 1,2,3-triazole | 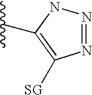 | H; SCH₃; COCH₃; Br; SOCH₃; SO₂CH₃; NO₂; CF₃; CN; COOMe |
| 1,2,4-triazole | 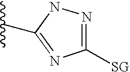 | H; SCH₃; COCH₃; Br; SOCH₃; SO₂CH₃; NO₂ |
| imidazole | 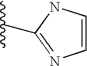 | H; SCH₃; COCH₃; Br; SOCH₃; SO₂CH₃; NO₂ |

Thus, isosteres include tetrazole, 1,2,3-triazole, 1,2,4-triazole and imidazole.

The compounds of formula (1) may be supplied in the form of their pharmaceutically acceptable acid-addition salts including salts of inorganic acids such as hydrochloric, sulfuric, hydrobromic, or phosphoric acid or salts of organic acids such as acetic, tartaric, succinic, benzoic, salicylic, and the like. If a carboxyl moiety is present on the compound of formula (1), the compound may also be supplied as a salt with a pharmaceutically acceptable cation.

Compounds useful in the practice of the disclosed invention include, but are not limited to, the compounds shown in Table 2, below.

TABLE 2
Exemplary p38 Inhibitors

| Cpd. # | Mol. Structure |
|---|---|
| 1 | 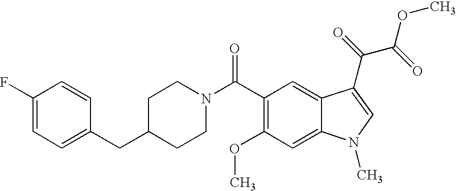 |
| 2 | 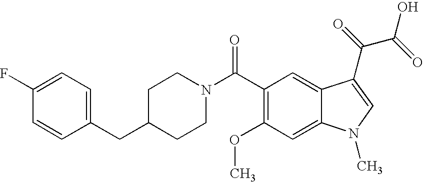 |
| 3 | 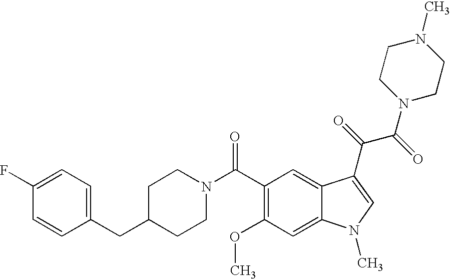 |
| 4 | 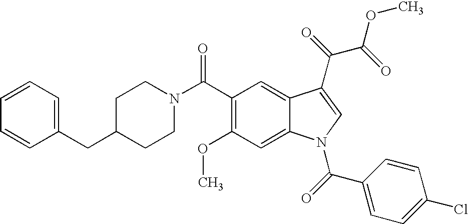 |
| 5 | 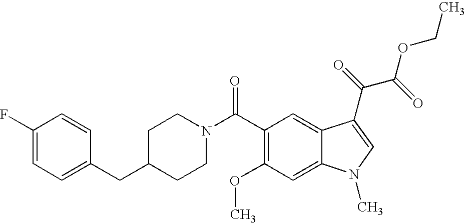 |
| 6 | 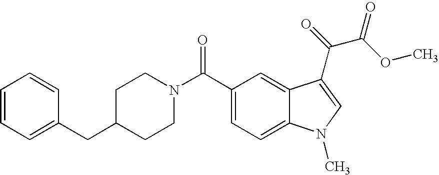 |
| 7 | 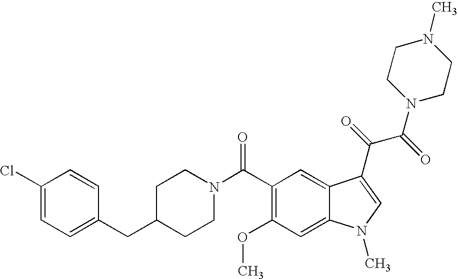 |
| 8 | 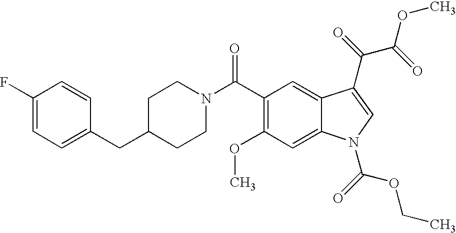 |
| 9 | 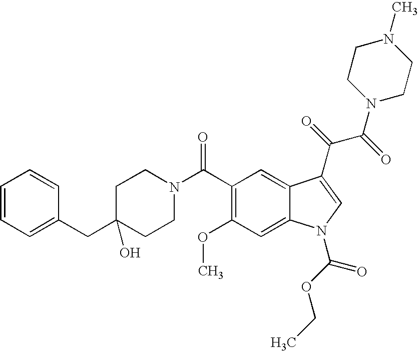 |
| 10 | 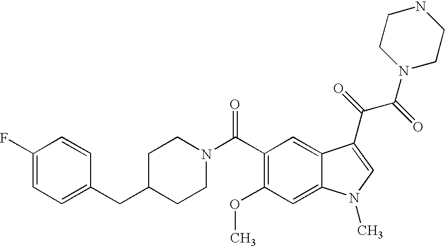 |
| 11 | 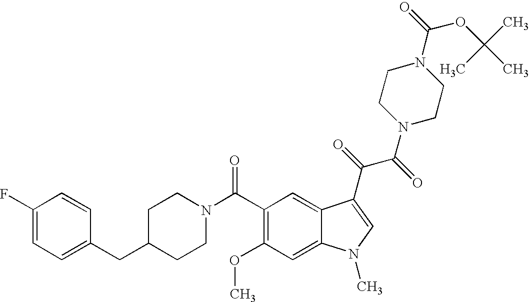 |
| 12 | 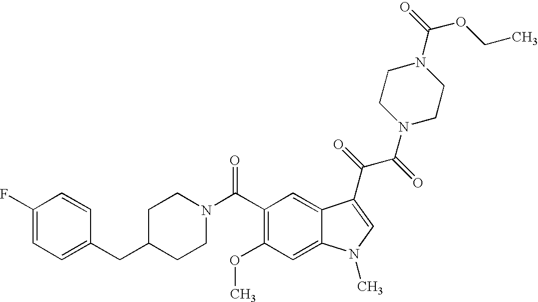 |
| 13 | 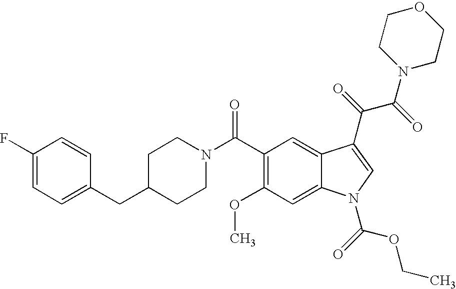 |
| 14 | 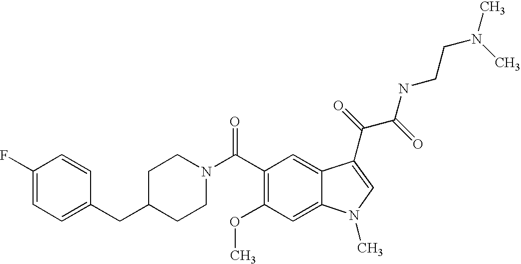 |
| 15 | 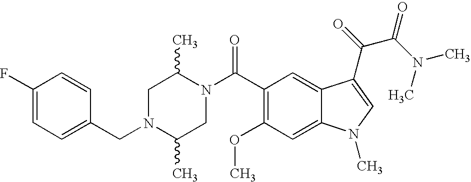 |
| 16 | 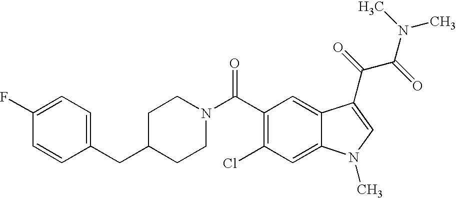 |
| 17 | 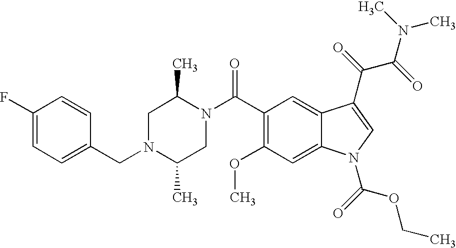 |
| 18 | 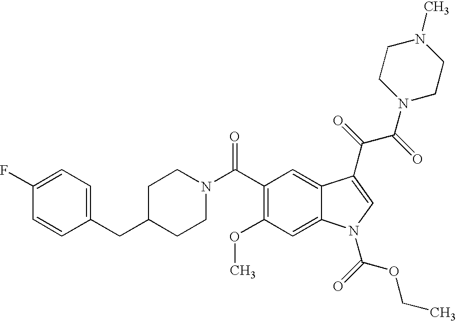 |
| 19 | 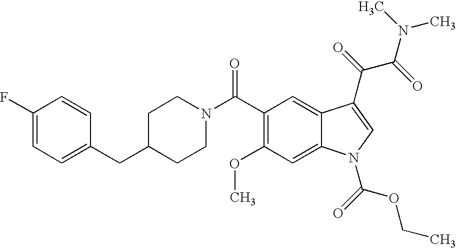 |
| 20 | 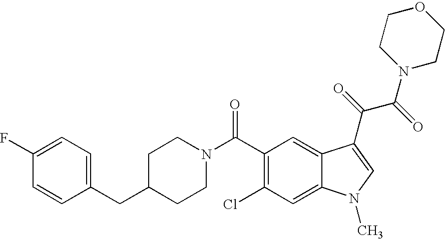 |
| 21 | 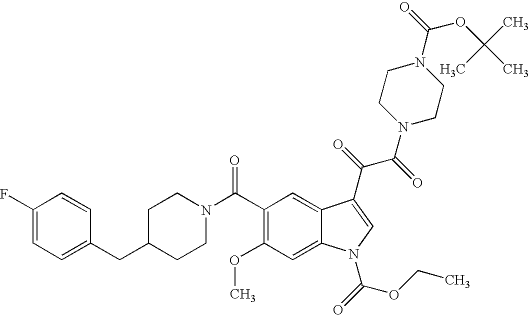 |
| 22 | 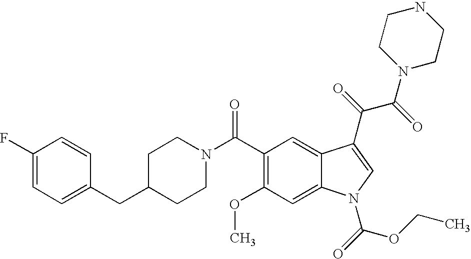 |
| 23 | 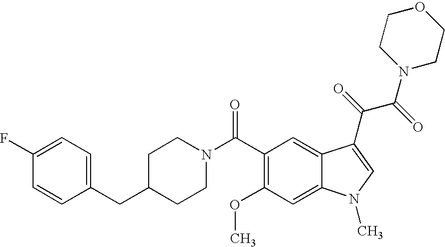 |
| 24 | 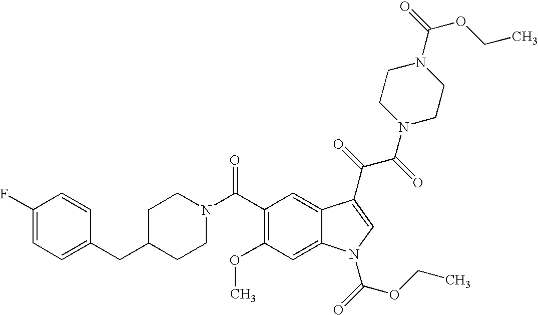 |
| 25 | 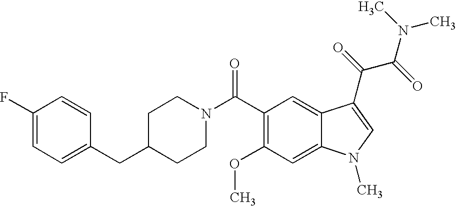 |
| 26 | 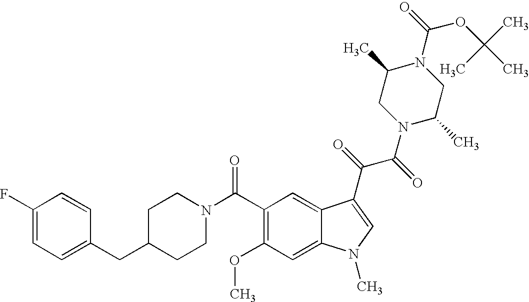 |
| 27 | 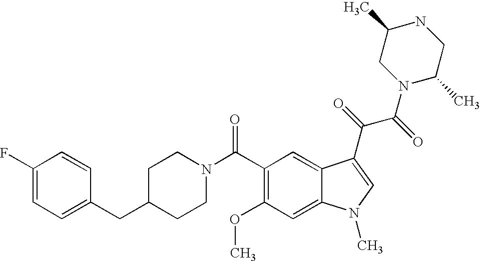 |
| 28 | 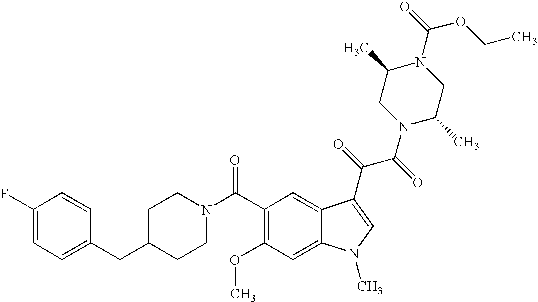 |
| 29 | 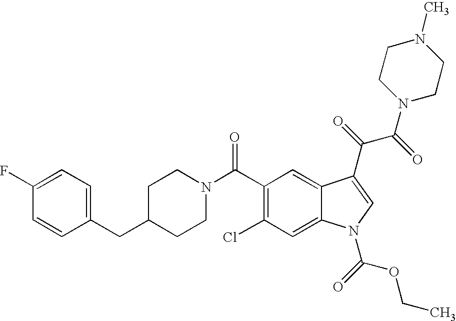 |
| 30 | 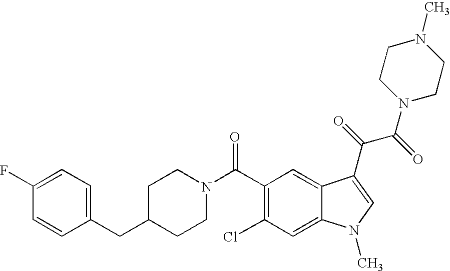 |
| 31 | 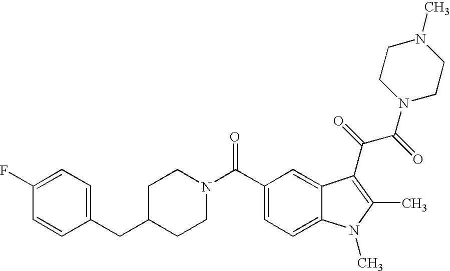 |
| 32 | 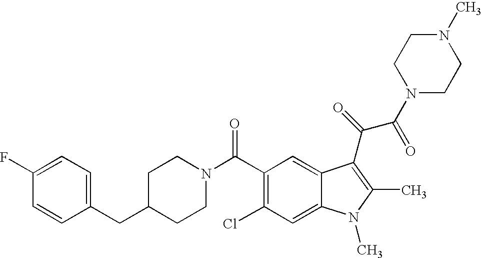 |
| 33 | 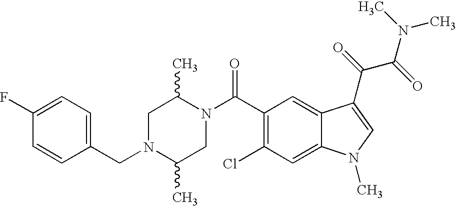 |
| 34 | 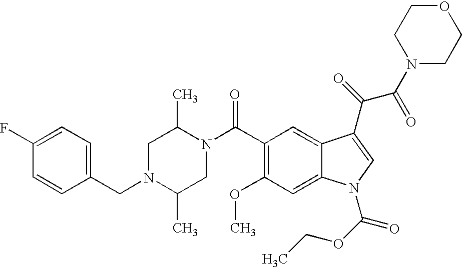 |
| 35 | 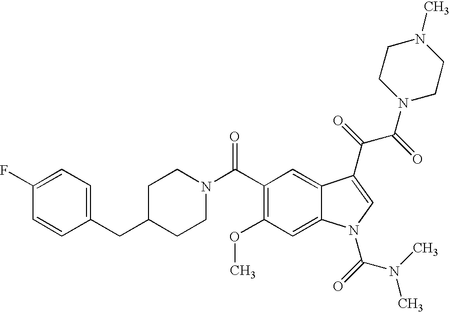 |
| 36 | 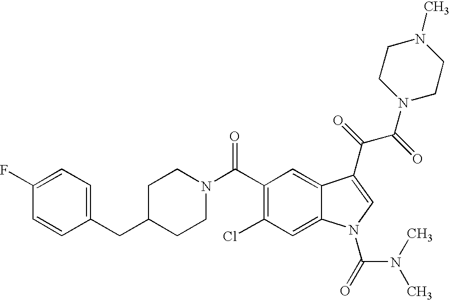 |
| 37 | 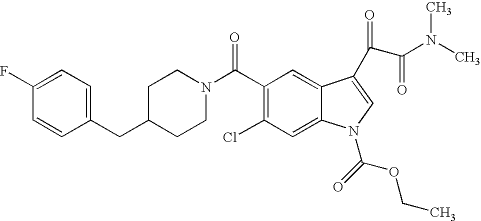 |
| 38 | 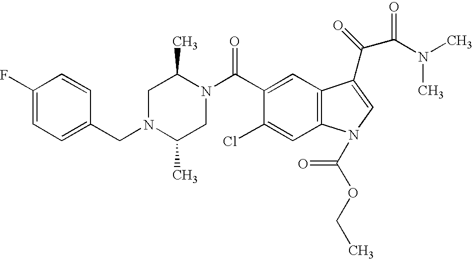 |
| 39 | 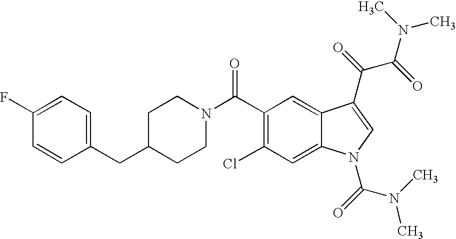 |
| 40 | 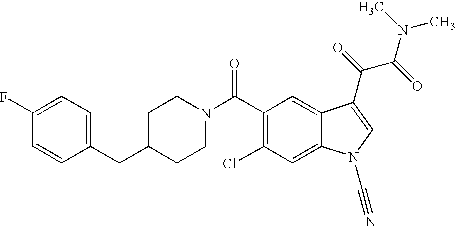 |
| 41 | 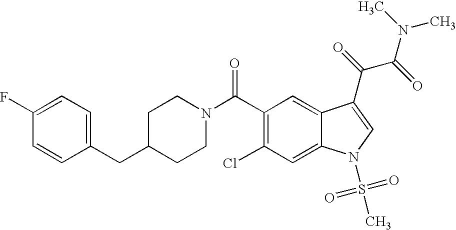 |
| 42 | 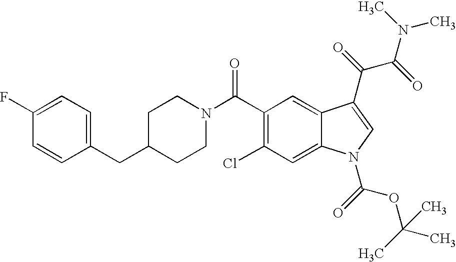 |
| 43 | 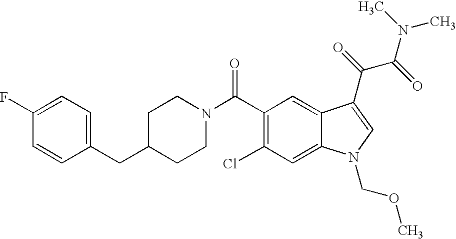 |
| 44 | 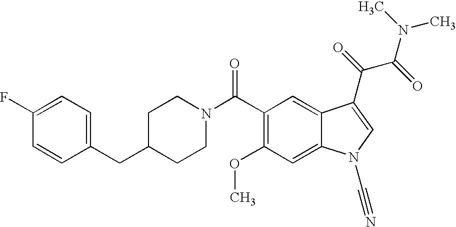 |
| 45 | 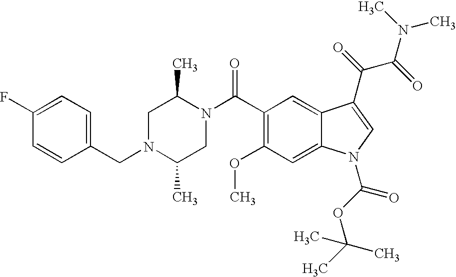 |
| 46 | 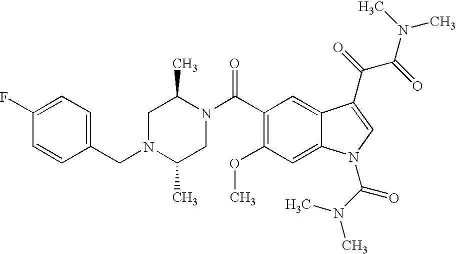 |
| 47 | 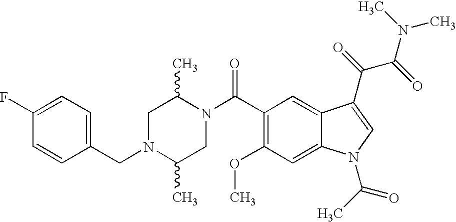 |
| 48 | 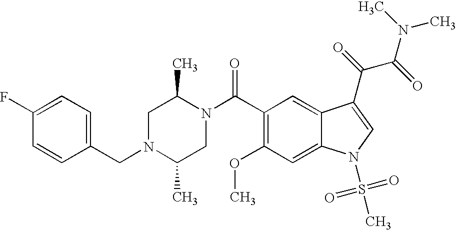 |
| 49 | 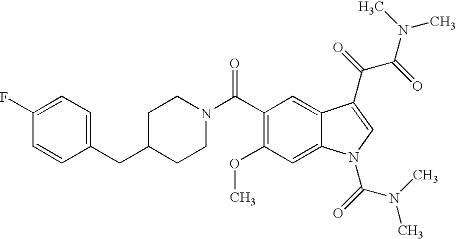 |
| 50 | 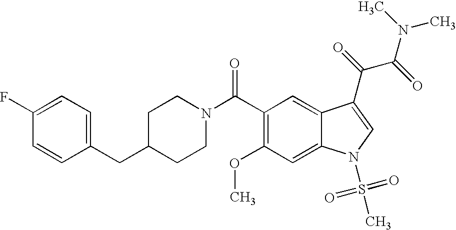 |
| 51 | 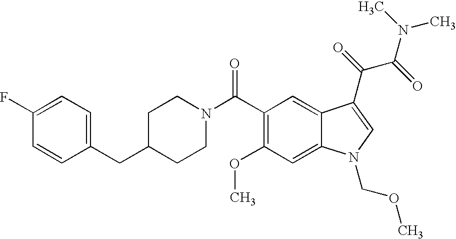 |
| 52 | 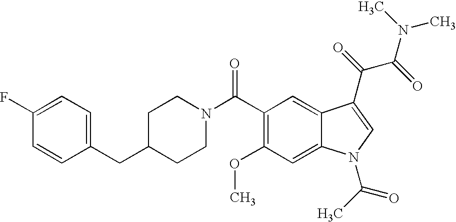 |
| 53 | 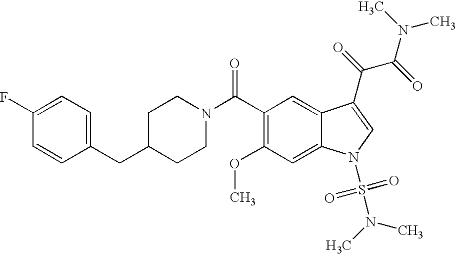 |
| 54 | 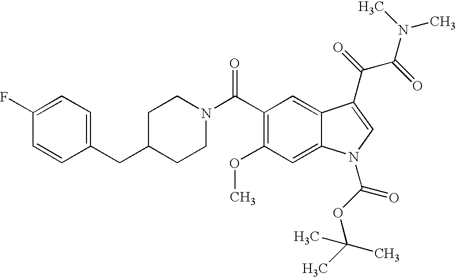 |
| 55 | 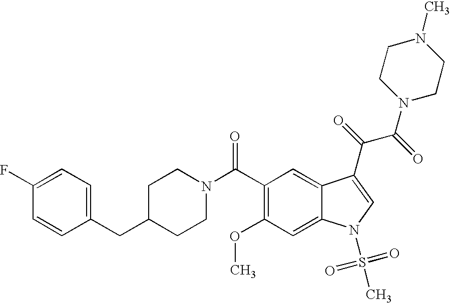 |
| 56 | 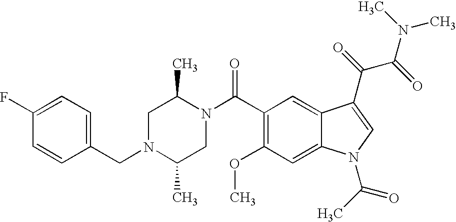 |
| 57 | 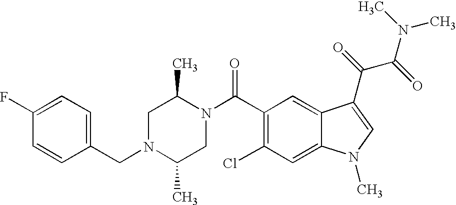 |
| 58 | 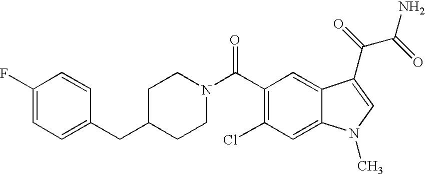 |
| 59 | 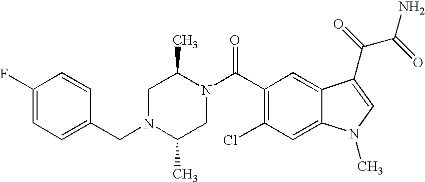 |
| 60 | 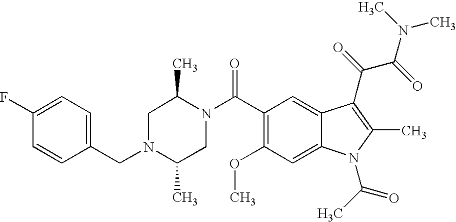 |
| 61 | 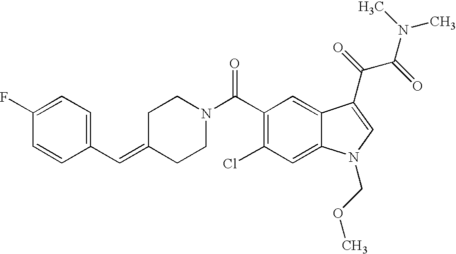 |
| 62 | 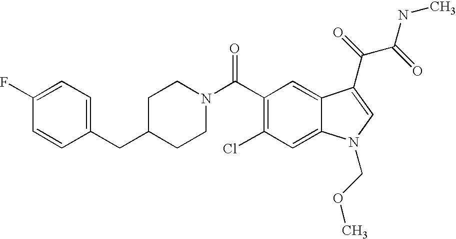 |
| 63 | 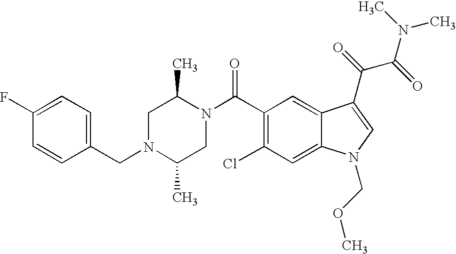 |
| 64 | 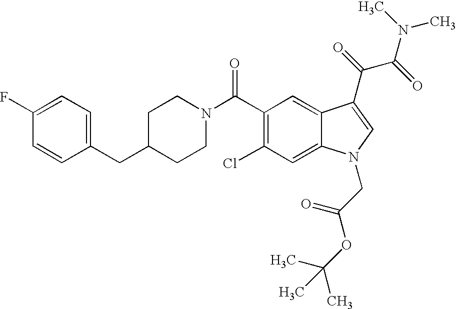 |
| 65 | 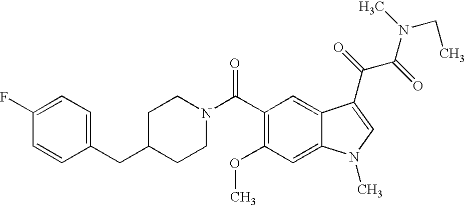 |
| 66 | 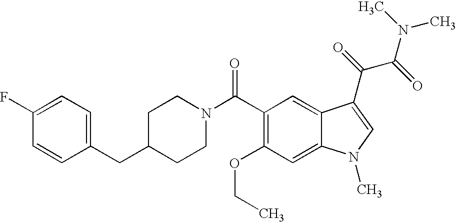 |
| 67 | 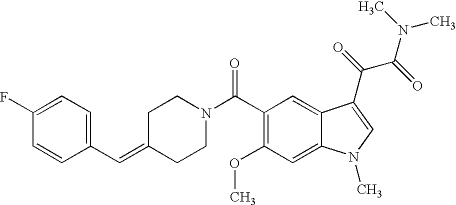 |
| 68 | 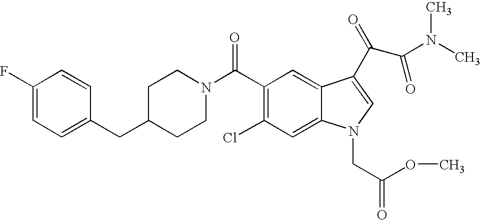 |
| 69 | 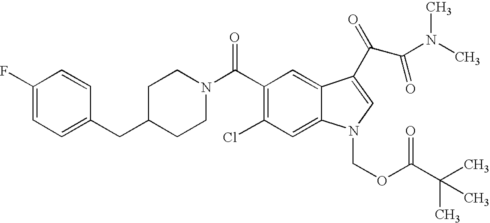 |
| 70 | 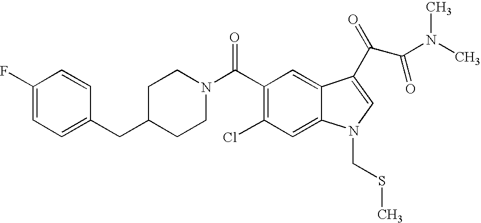 |
| 71 | 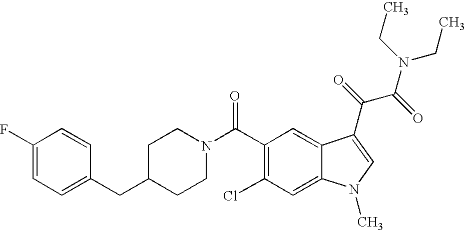 |
| 72 | 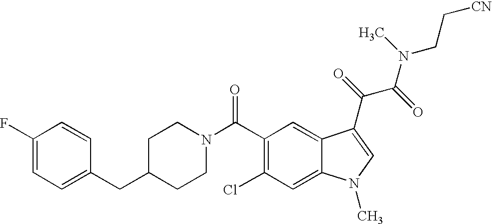 |
| 73 | 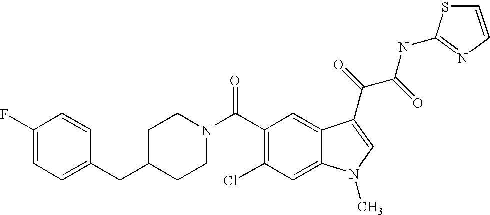 |
| 74 | 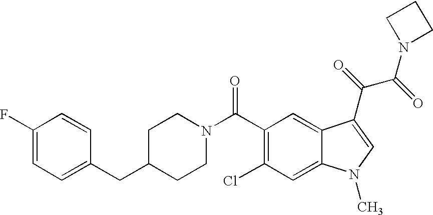 |
| 75 | 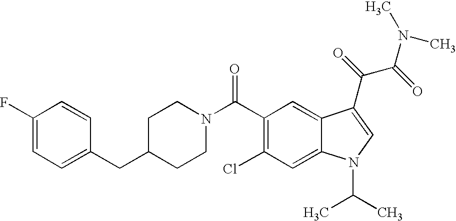 |
| 76 | 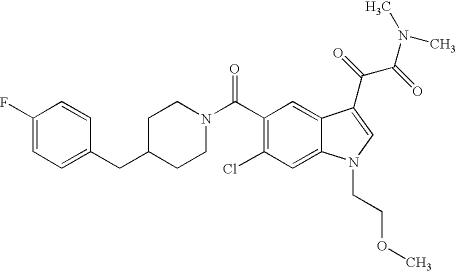 |
| 77 | 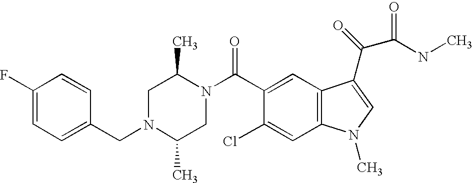 |
| 78 | 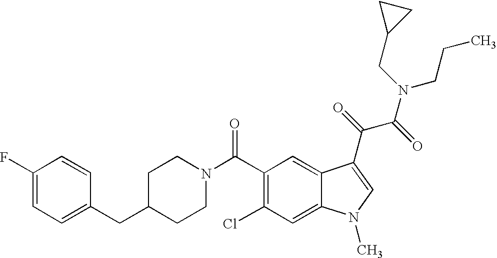 |
| 79 | 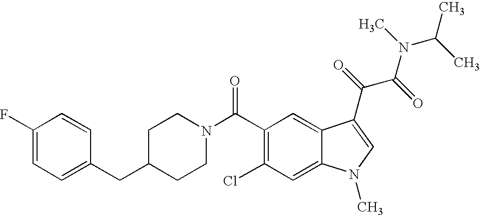 |
| 80 | 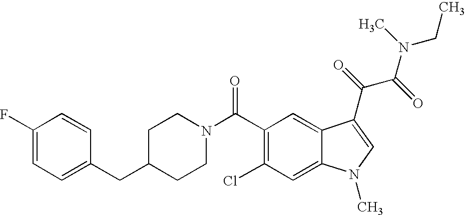 |
| 81 | 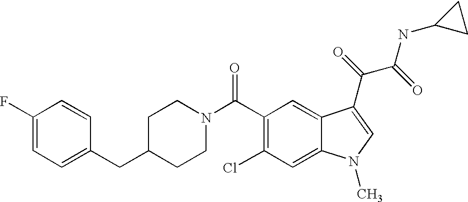 |
| 82 | 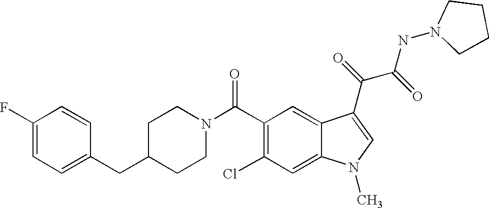 |
| 83 | 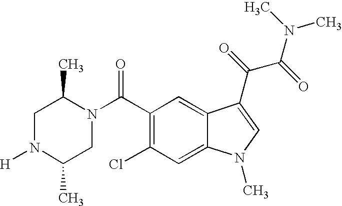 |
| 84 | 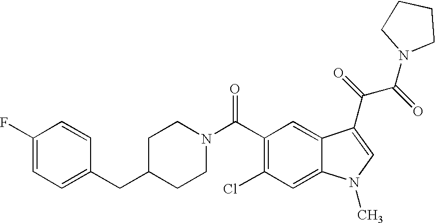 |
| 85 | 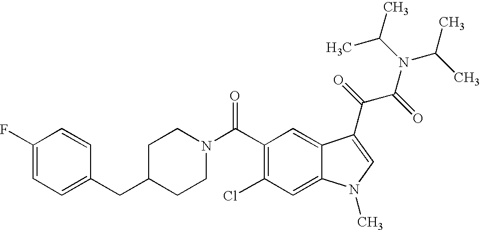 |
| 86 | 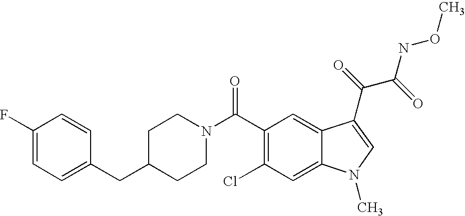 |
| 87 | 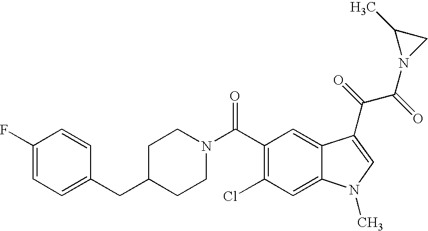 |
| 88 | 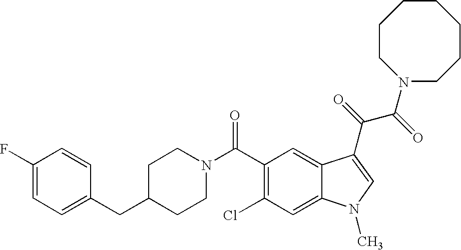 |
| 89 | 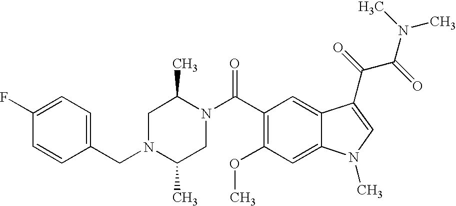 |
| 90 | 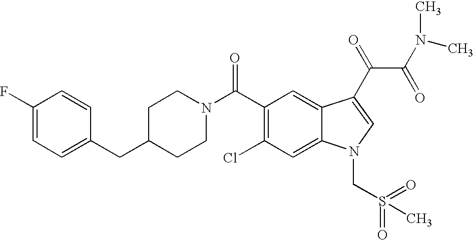 |
| 91 | 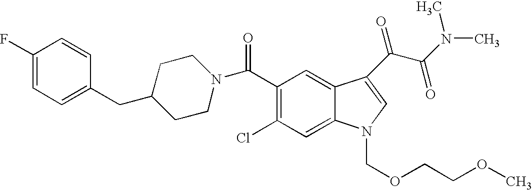 |
| 92 | 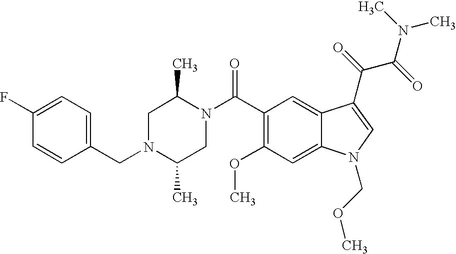 |
| 93 | 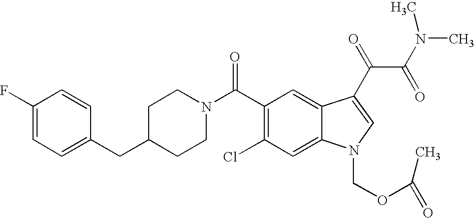 |
| 94 | 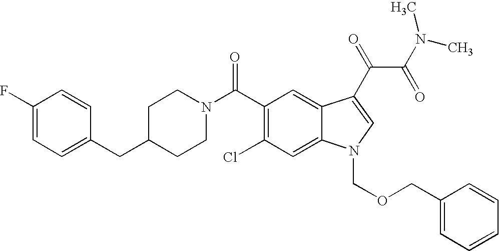 |
| 95 | 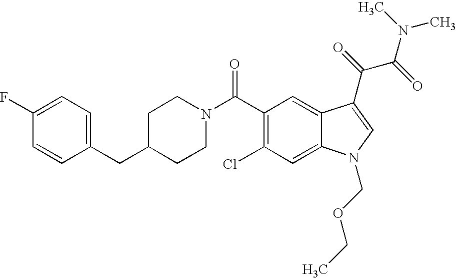 |
| 96 | 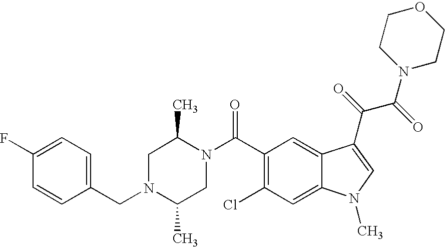 |
| 97 | 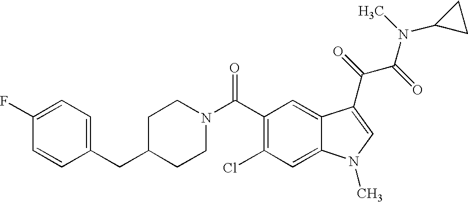 |
| 98 | 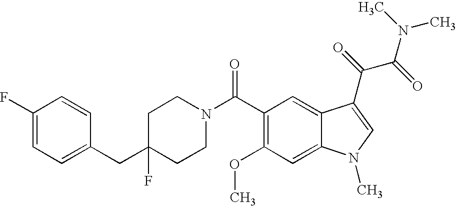 |
| 99 | 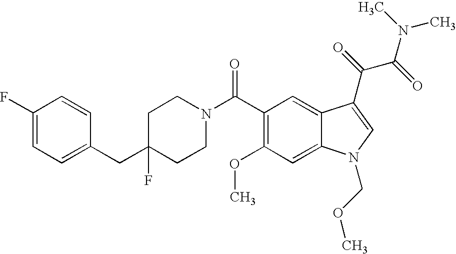 |
| 100 | 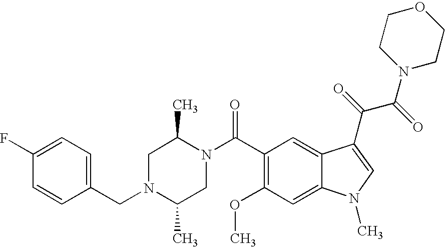 |
| 101 | 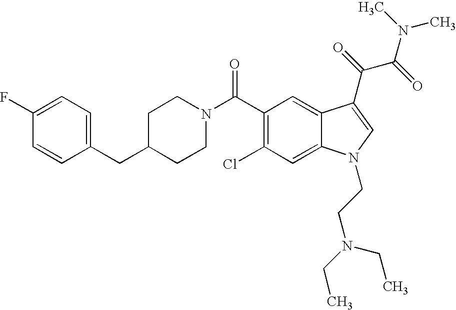 |
| 102 | 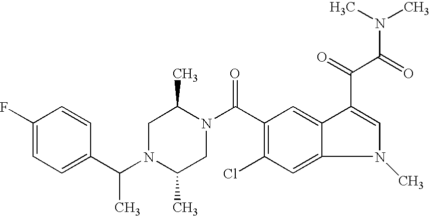 |
| 103 | 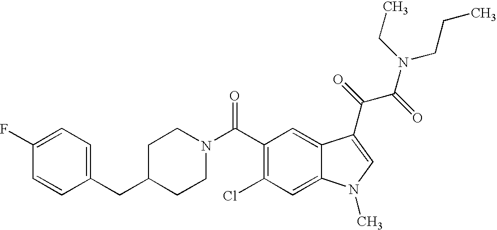 |
| 104 | 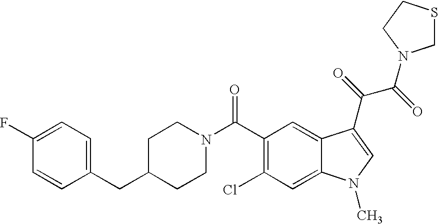 |
| 105 | 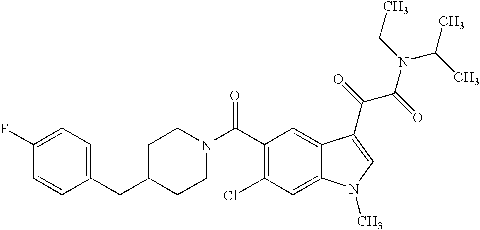 |
| 106 | 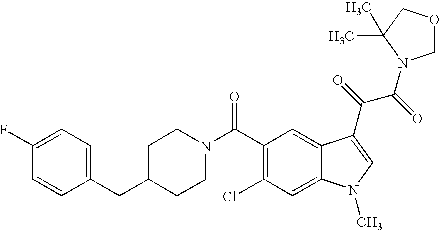 |
| 107 | 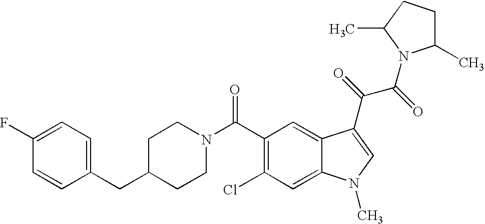 |
| 108 | 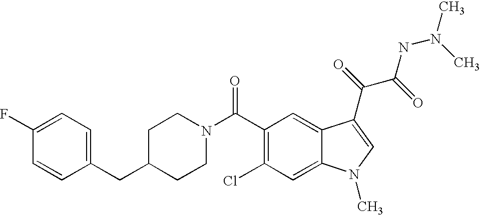 |
| 109 | 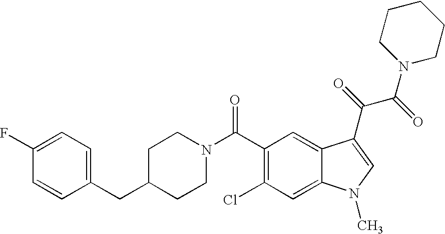 |
| 110 | 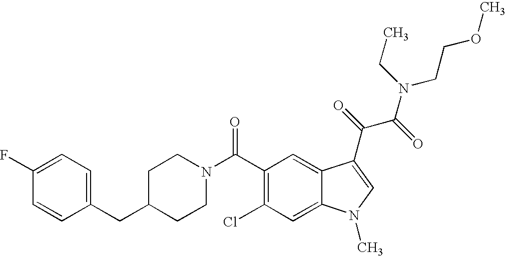 |
| 111 | 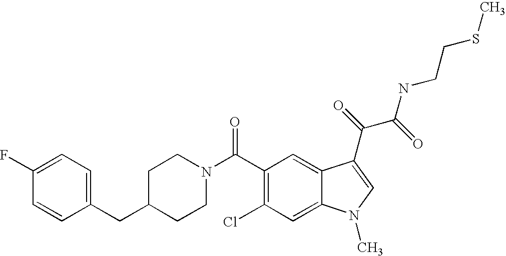 |
| 112 | 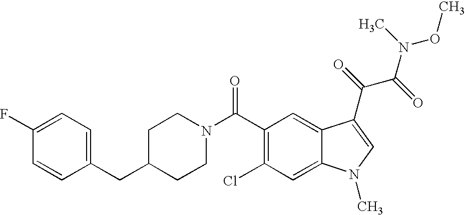 |
| 113 | 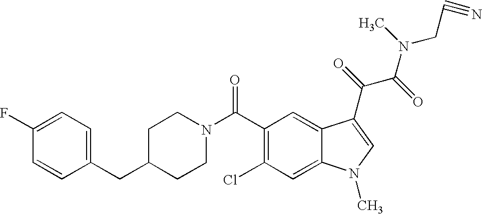 |
| 114 | 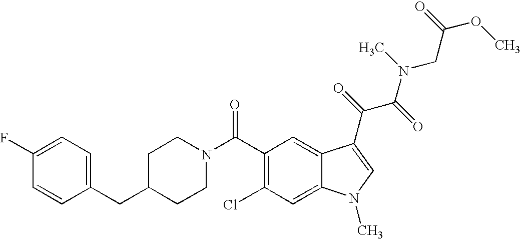 |
| 115 | 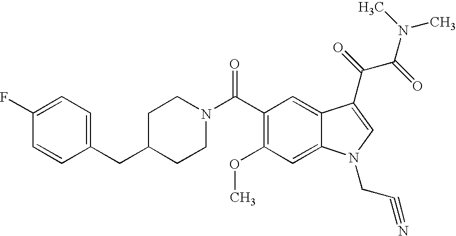 |
| 116 | 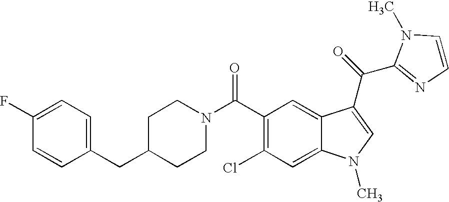 |
| 117 | 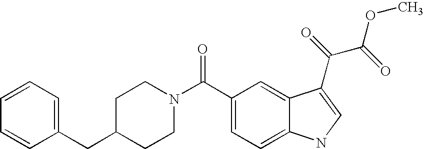 |
| 118 | 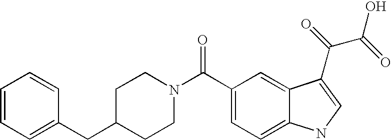 |
| 119 | 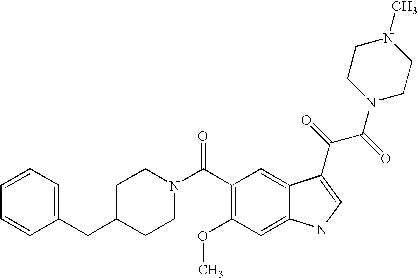 |
| 120 | 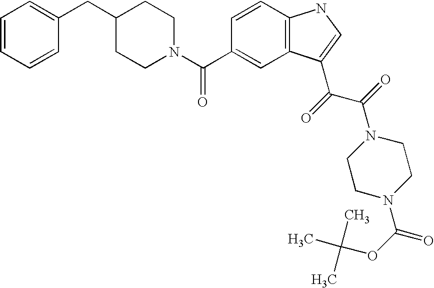 |
| 121 | 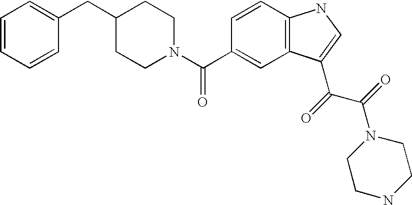 |
| 122 | 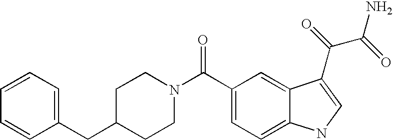 |
| 123 | 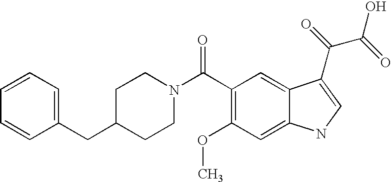 |
| 124 | 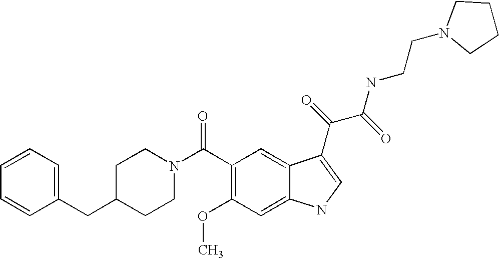 |
| 125 | 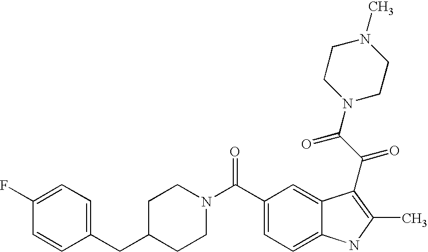 |
| 126 | 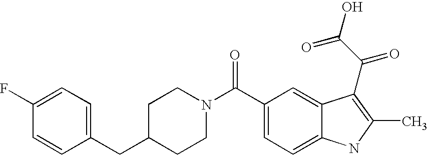 |
| 127 | 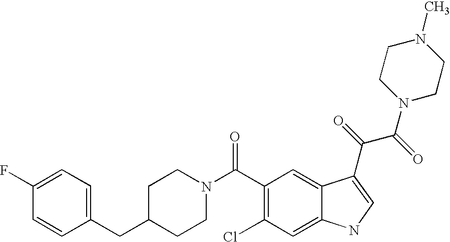 |
| 128 | 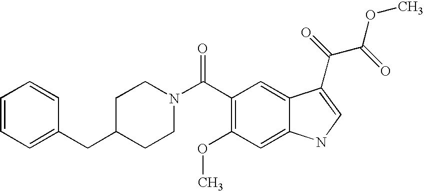 |
| 129 | 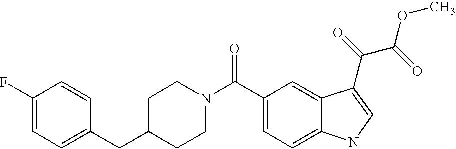 |
| 130 | 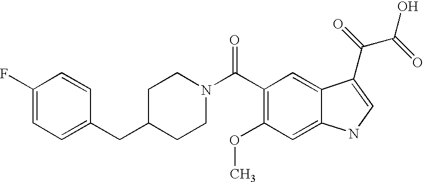 |
| 131 | 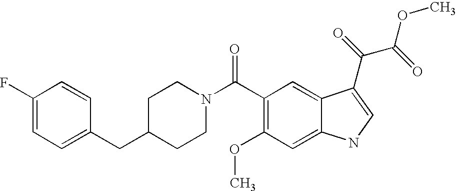 |
| 132 | 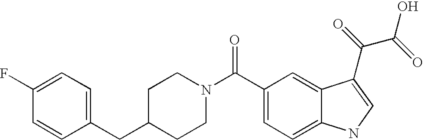 |
| 133 | 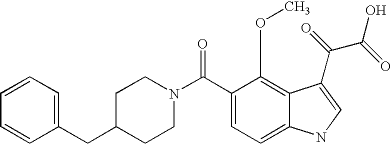 |
| 134 | 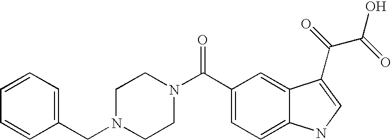 |
| 135 | 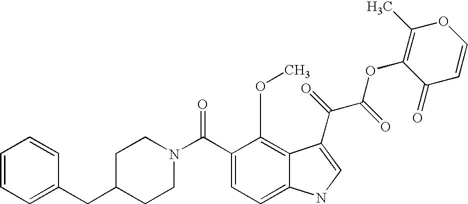 |
| 136 | 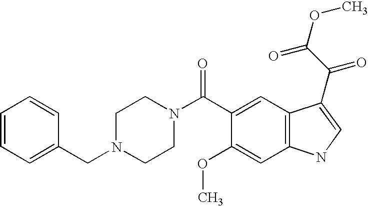 |
| 137 | 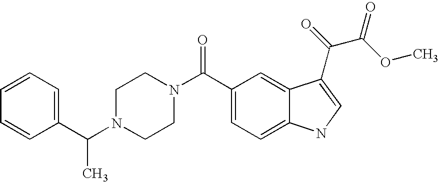 |
| 138 | 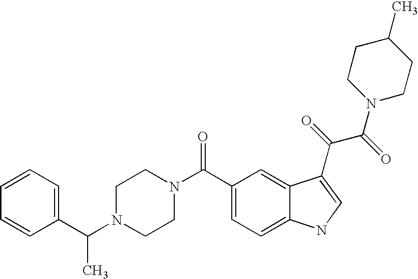 |
| 139 | 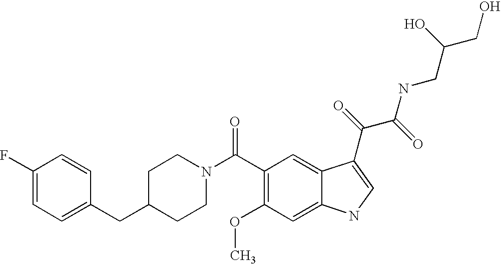 |
| 140 | 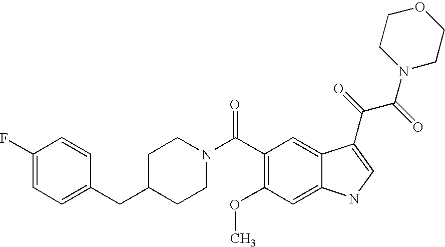 |
| 141 | 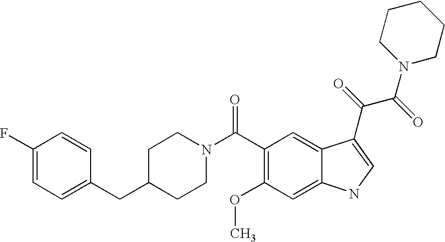 |
| 142 | 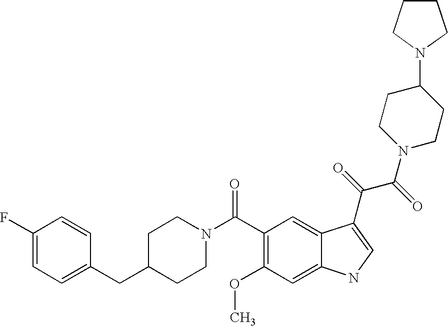 |
| 143 | 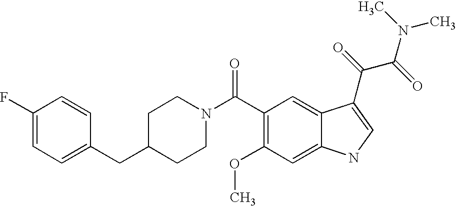 |
| 144 | 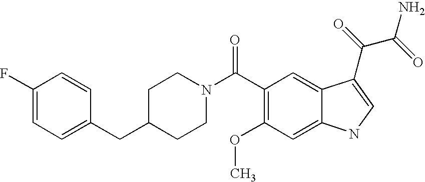 |
| 145 | 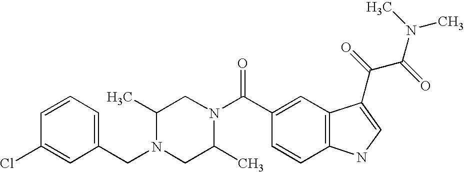 |
| 146 | 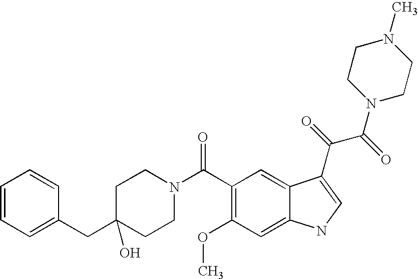 |
| 147 | 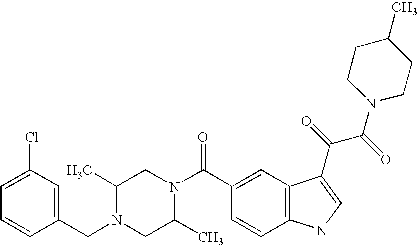 |
| 148 | 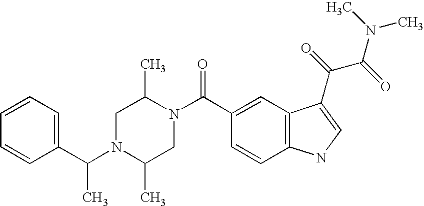 |
| 149 | 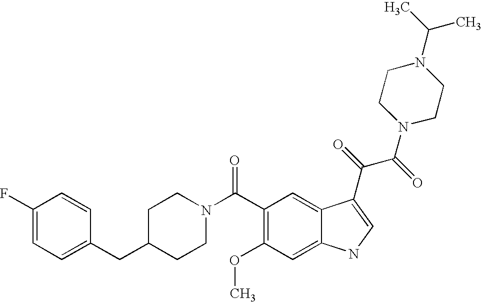 |
| 150 | 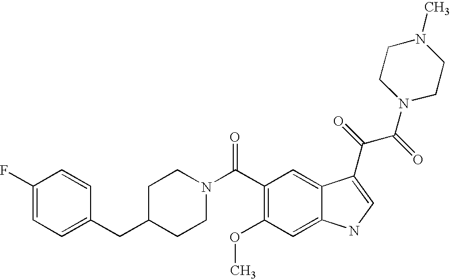 |
| 151 | 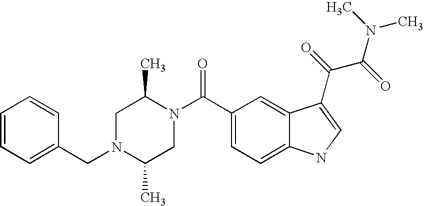 |
| 152 | 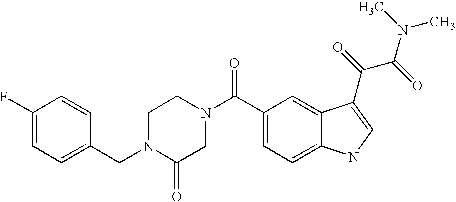 |
| 153 | 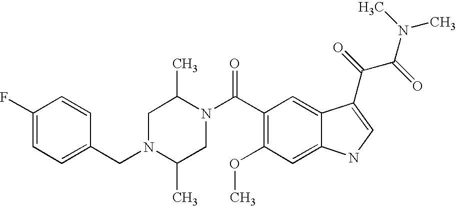 |
| 154 | 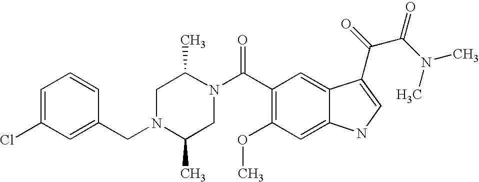 |
| 155 | 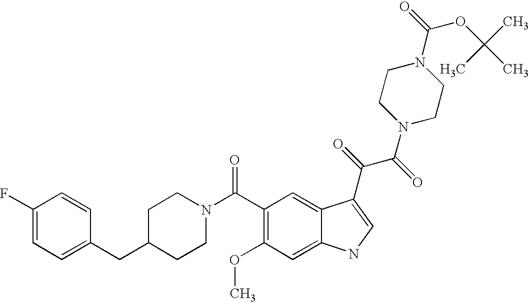 |
| 156 | 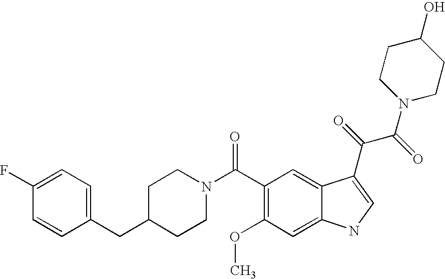 |
| 157 | 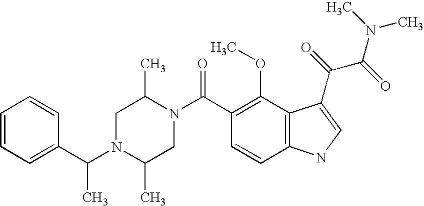 |
| 158 | 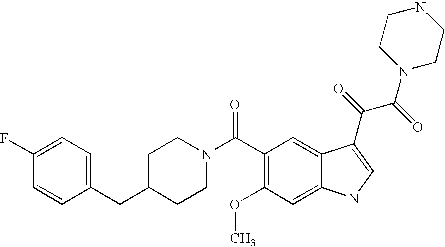 |
| 159 | 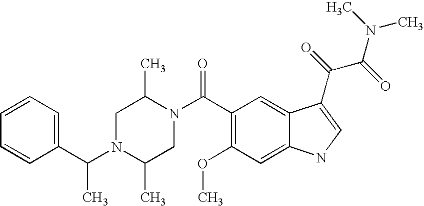 |
| 160 | 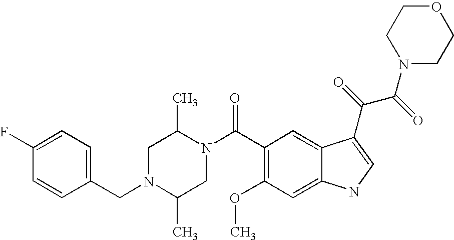 |
| 161 | 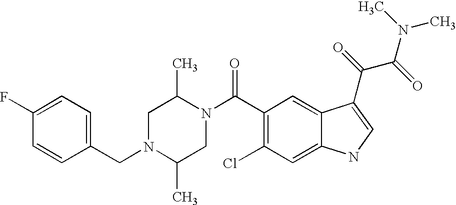 |
| 162 | 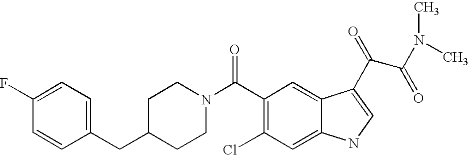 |
| 163 | 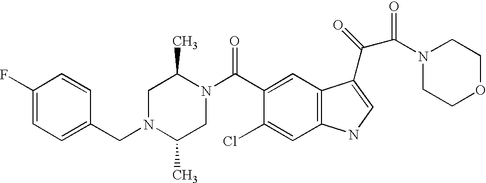 |
| 164 | 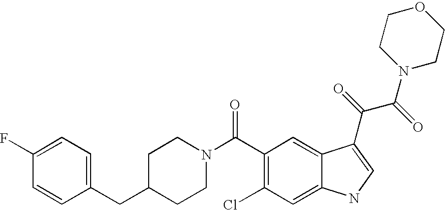 |
| 165 | 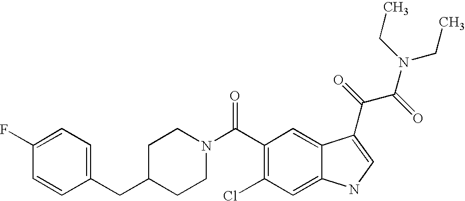 |
| 166 | 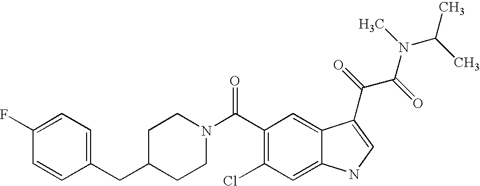 |
| 167 | 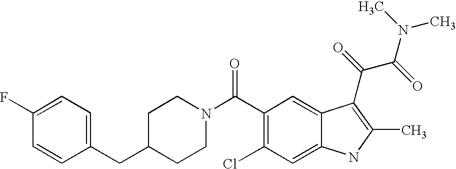 |
| 168 | 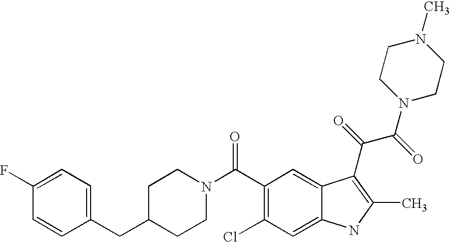 |
| 169 | 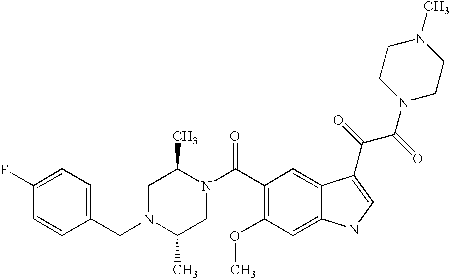 |
| 170 | 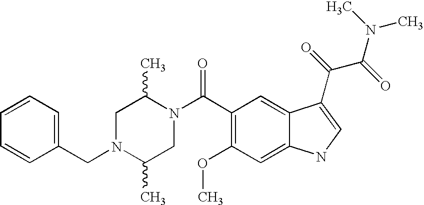 |
| 171 | 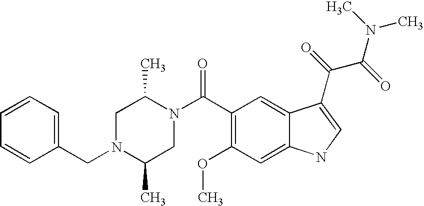 |
| 172 | 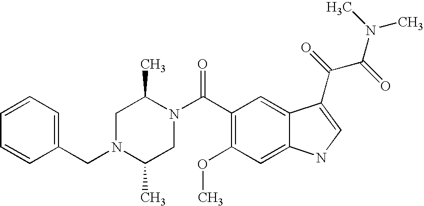 |
| 173 | 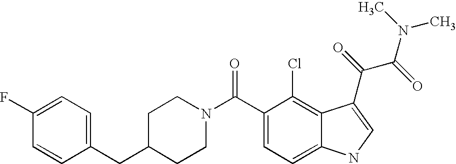 |
| 174 | 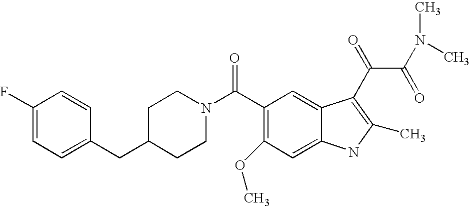 |
| 175 | 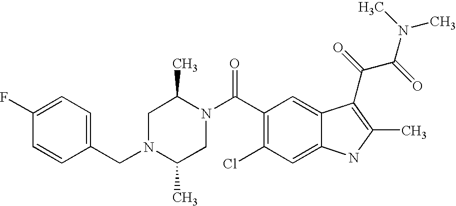 |
| 176 | 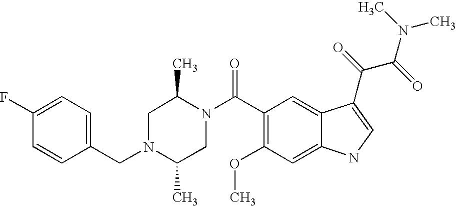 |
| 177 | 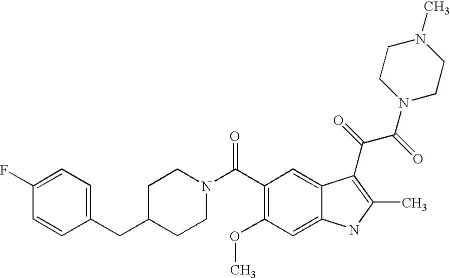 |
| 178 | 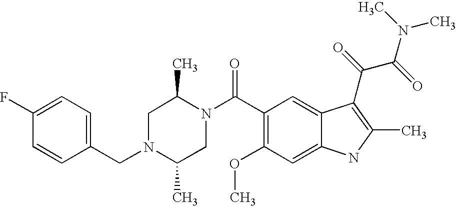 |
| 179 | 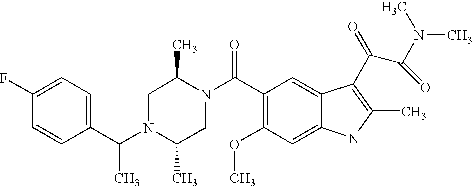 |
| 180 | 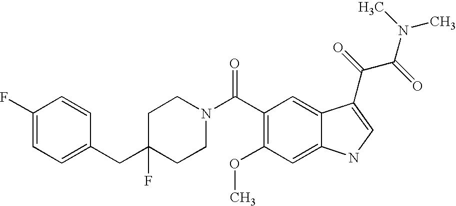 |
| Sigma Compound Product Number S8307 | 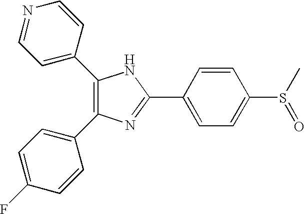 |

Additional compounds are described in published PCT applications WO 96/21452, WO 96/40143, WO 97/25046, WO 97/35856, WO 98/25619, WO 98/56377, WO 98/57966, WO 99/32110, WO 99/32121, WO 99/32463, WO 99/61440, WO 99/64400, WO 00/10563, WO 00/17204, WO 00/19824, WO 00/41698, WO 00/64422, WO 00/71535, WO 01/38324, WO 01/64679, WO 01/66539, and WO 01/66540, each of which is herein incorporated by reference in their entirety.

Further additional compounds useful in the practice of the present invention also include, but are not limited to, the compounds shown in Table 3, below.

TABLE 3

| | Citations, each of which is herein |
|---|---|
| Chemical Structure | incorporated by reference. |
| 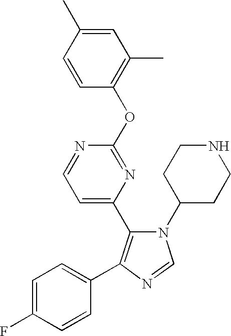 | WO-00166539, WO-00166540, WO-00164679, WO-00138324, WO-00064422, WO-00019824, WO-00010563, WO-09961440, WO-09932121, WO-09857966, WO-09856377, WO-09825619, WO-05756499, WO-09735856, WO-09725046, WO-09640143, WO-09621452; Gallagher, T.F., et. Al., Bioorg. Med. Chem. 5:49 (1997); Adams, J.L., et al., Bioorg. Med. Chem. Lett. 8:3111-3116 (1998) |
| 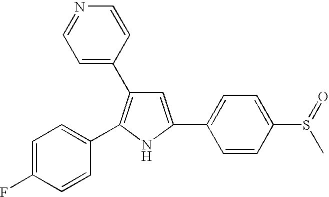 | De Laszlo, S.E., et. Al., Bioorg Med Chem Lett. 8:2698 (1998) |
| 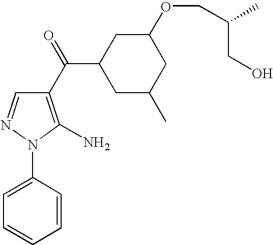 | WO-09957101; Poster presentation at the 5^{th} World Congress on Inflammation, Edinburgh, UK. (2001) |
| 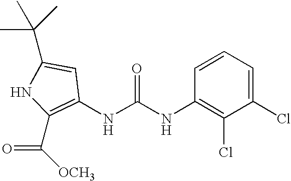 | WO-00041698, WO-09932110, WO-09932463 |
| 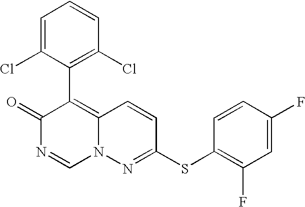 | WO-00017204, WO-09964400 |
| 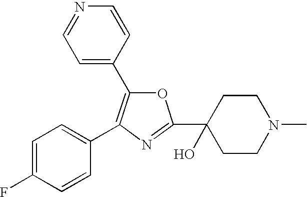 | Revesz. L., et. al., Bioorg Med Chem Lett. 10:1261 (2000) |
| 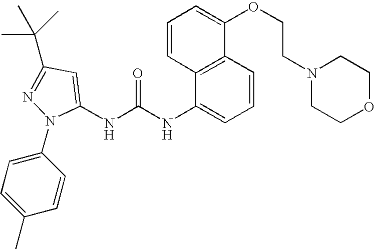 | WO-00207772 |
| 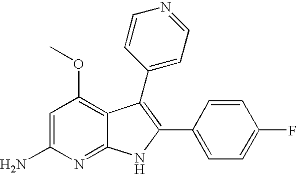 | Fijen, J.W., et al., Clin. Exp. Immunol. 124:16-20 (2001); Wadsworth, S.A., et. al., J. Pharmacol. Expt. Therapeut. 291:680 (1999) |
| 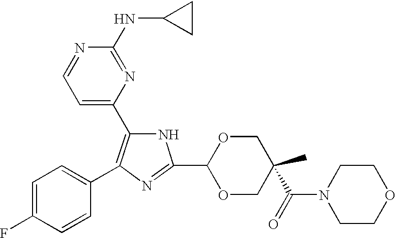 | Collis, A.J., et al.. Bioorg. Med Chem. Lett. 11:693-696 (2001); McLay, L.M., et al., Bioorg Med Chem 9:537-554 (2001) |
| 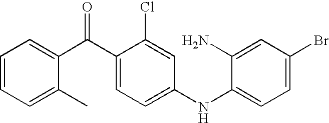 | WO-00110865, WO-00105749 |

Additional guidance regarding p38 MAPK inhibitory compounds is found in U.S. patent application Ser. No. 09/575,060 (now U.S. Pat. No. 6,867,209), Ser. No. 10/157,048 (now U.S. Pat. No. 6,864,260), Ser. Nos. 10/146,703, 10/156,997, and 10/156,996, all of which are hereby incorporated by reference in their entirety. The compounds described above are provided for guidance and exemplary purposes only. It should be understood that any modulator of a p38MAP kinase that plays a role in the genesis and maintenance of the MM disease state is useful for the invention provided that it exhibits adequate activity relative to the targeted protein.

Utility and Administration

The methods and compositions of the invention are successful to treat or ameliorate osteolytic lesions caused or induced by multiple myeloma in humans.

As used herein, “treat” or “treatment” include effecting postponement of development of undesirable conditions and/or reduction in the severity of such symptoms that will or are expected to develop. Treatment includes ameliorating existing symptoms, preventing additional symptoms, ameliorating or preventing the underlying metabolic causes of symptoms, preventing the severity of the condition or reversing the condition, at least partially. Thus, the terms denote that a beneficial result has been conferred on a subject with multiple myeloma.

Treatment generally comprises “administering” to a subject a compound which includes providing the subject compound in a therapeutically effective amount. “Therapeutically effective amount” means the amount of the compound that will treat multiple myeloma related osteolytic lesions by eliciting a favorable response in a cell, tissue, organ, system, in a human. The response may be preventive or therapeutic. The administering may be of the compound per se in a pharmaceutically acceptable composition, or this composition may include combinations with other active ingredients that are suitable to the treatment of this condition. The compounds may be administered in a prodrug form.

The manner of administration and formulation of the compounds useful in the invention and their related compounds will depend on the composition of the compound, the nature of the condition, the severity of the condition, the particular subject to be treated, and the judgment of the practitioner; formulation will also depend on mode of administration. For example, if the compounds are “small molecules,” they might be conveniently administered by oral administration by compounding them with suitable pharmaceutical excipients so as to provide tablets, capsules, syrups, and the like. Suitable formulations for oral administration may also include minor components such as buffers, flavoring agents and the like. Typically, the amount of active ingredient in the formulations will be in the range of 5%-95% of the total formulation, but wide variation is permitted depending on the carrier. Suitable carriers include sucrose, pectin, magnesium stearate, lactose, peanut oil, olive oil, water, and the like. This method is preferred if the subject can tolerate oral administration.

The compounds useful in the invention may also be administered through suppositories or other transmucosal vehicles. Typically, such formulations will include excipients that facilitate the passage of the compound through the mucosa such as pharmaceutically acceptable detergents.

The compounds may also be administered topically, or in formulation intended to penetrate the skin. These include lotions, creams, ointments and the like which can be formulated by known methods.

The compounds may also be administered by injection, including intravenous, intramuscular, subcutaneous or intraperitoneal injection. Typical formulations for such use are liquid formulations in isotonic vehicles such as Hank's solution or Ringer's solution.

Intravenous administration is preferred for acute conditions; generally in these circumstances, the subject will be hospitalized. The intravenous route avoids any problems with inability to absorb the orally administered drug.

Alternative formulations include nasal sprays, liposomal formulations, slow-release formulations, and the like, as are known in the art.

Any suitable formulation may be used. A compendium of art-known formulations is found in Remington's Pharmaceutical Sciences, latest edition, Mack Publishing Company, Easton, Pa. Reference to this manual is routine in the art.

Thus, the compounds useful in the method of the invention may be administered systemically or locally. For systemic use, the compounds are formulated for parenteral (e.g., intravenous, subcutaneous, intramuscular, intraperitoneal, intranasal or transdermal) or enteral (e.g., oral or rectal) delivery according to conventional methods. Intravenous administration can be by a series of injections or by continuous infusion over an extended period. Administration by injection or other routes of discretely spaced administration can be performed at intervals ranging from weekly to once to three times daily. Alternatively, the compounds may be administered in a cyclical manner (administration of compound; followed by no administration; followed by administration of compound, and the like). Treatment will continue until the desired outcome is achieved. In general, pharmaceutical formulations will include an active ingredient in combination with a pharmaceutically acceptable vehicle, such as saline, buffered saline, 5% dextrose in water, borate-buffered saline containing trace metals or the like. Formulations may further include one or more excipients, preservatives, solubilizers, buffering agents, albumin to prevent protein loss on vial surfaces, lubricants, fillers, stabilizers, etc.

Pharmaceutical compositions can be in the form of sterile, non-pyrogenic liquid solutions or suspensions, coated capsules, suppositories, lyophilized powders, transdermal patches or other forms known in the art.

Biodegradable films or matrices may be used in the invention methods. These include calcium sulfate, tricalcium phosphate, hydroxyapatite, polylactic acid, polyanhydrides, bone or dermal collagen, pure proteins, extracellular matrix components and the like and combinations thereof. Such biodegradable materials may be used in combination with non-biodegradable materials, to provide desired mechanical, cosmetic or tissue or matrix interface properties.

Alternative methods for delivery may include osmotic minipumps; sustained release matrix materials such as electrically charged dextran beads; collagen-based delivery systems, for example; methylcellulose gel systems; alginate-based systems, and the like.

Aqueous suspensions may contain the active ingredient in admixture with pharmacologically acceptable excipients, comprising suspending agents, such as methyl cellulose; and wetting agents, such as lecithin, lysolecithin or long-chain fatty alcohols. The said aqueous suspensions may also contain preservatives, coloring agents, flavoring agents, sweetening agents and the like in accordance with industry standards.

Preparations for topical and local application comprise aerosol sprays, lotions, gels and ointments in pharmaceutically appropriate vehicles which may comprise lower aliphatic alcohols, polyglycols such as glycerol, polyethylene glycol, esters of fatty acids, oils and fats, and silicones. The preparations may further comprise antioxidants, such as ascorbic acid or tocopherol, and preservatives, such as p-hydroxybenzoic acid esters.

Parenteral preparations comprise particularly sterile or sterilized products. Injectable compositions may be provided containing the active compound and any of the well known injectable carriers. These may contain salts for regulating the osmotic pressure.

Liposomes may also be used as a vehicle, prepared from any of the conventional synthetic or natural phospholipid liposome materials including phospholipids from natural sources such as egg, plant or animal sources such as phosphatidylcholine, phosphatidylethanolamine, phosphatidylglycerol, sphingomyelin, phosphatidylserine, or phosphatidylinositol and the like. Synthetic phospholipids may also be used.

The dosages of the compounds of the invention will depend on a number of factors which will vary from subject to subject. However, it is believed that generally, the daily dosage in humans (average weight of 70 kg) will range between 30 mg and 500 mg, preferably between 45 mg and 400 mg, more preferably between 50 mg and 300 mg per day. The dose regimen will vary, however, depending on the compound and formulation selected, the condition of the subject being treated and the judgment of the practitioner. Optimization of dosage, formulation and regimen is routine for practitioners of the art.

The following examples are intended to illustrate but not to limit the invention.

### EXAMPLES

The following examples describe experiments to evaluate the effectiveness of p38 MAPK inhibitors as a treatment for multiple myeloma in a patient in need thereof. Table 2 lists a number of compounds that generally exhibit p38 MAPK activity, preferred embodiments exhibit a relative IC₅₀ value of less than 5 nM in an assay similar to the phosphorylation assay disclosed above (see Kumar). The compounds listed in Table 2 exemplify the compounds generically disclosed herein. Moreover, the data discussed below is representative of the genus of p38 MAPK inhibitors disclosed herein. The results discussed below are thought to be obtainable using any of the p38 MAPK inhibitors disclosed herein. As such, the data provided demonstrates that the genus of p38MAPK inhibitor compounds disclosed herein are useful in the disclosed methods of treating multiple myeloma. The Sigma-Aldrich® under product number S8307 compound is 4-(4-Fluorophenyl)-2-(4-methylsulfinylphenyl)-5-(4-pyridyl)-1H-imidazole, which is known in the literature as a p38 MAPK modulator and commercial available. This compound is available as a positive control in a p38 MAPK inhibition assay.

### Example 1

### p38 Activation in Multiple Myeloma

p38 MAPK is activated via dual phosphorylation by MEKK3 and/or MEKK6. The activated form of p38α MAPK is found in untreated and TNFα-activated MM cells, and in BMSC, using p38α phospho-specific immunodetection. BMSC are obtained from seven different donors (three normal healthy individuals and four MM patients). In the experiments reported here, no differences between BMSCs obtained from patients and healthy individuals were noted. A variety of widely used MM cell lines were also used in the studies described below.

The phosphorylation of p38α MAPK in MM cells was substantially suppressed by the MAPK inhibitor Compound 57 shown in Table 2 (see FIG. 1 and FIG. 2), while the phosphorylation of p38α MAPK in BMSC was partially suppressed (FIG. 3). This inhibitor blocked activity of p38 MAPK, but not the direct activation of the p38 MAPK enzyme or the activity of kinases upstream of p38 MAPK (e.g., MKK3 and MKK6); therefore this cellular effect is presumed to result from disruption of a feedback loop involving p38 MAPK kinase activity. As expected, p38 MAPK activity was fully suppressed by the p38 MAPK inhibitor, shown by immunodetection of p38 MAPK kinase target HSP-27. The Compound 57 blocked phosphorylation of HSP-27 completely in MM cells and in BMSC.

Neither p38 MAPK inhibition nor high concentrations of p38 MAPK inhibitor (tested up to 50-fold excess of active concentration) affected BMSC viability (FIG. 4). Viability was measured using a standard enzymatic assay of respiratory activity, MTS (3-(4,5-dimethylthiazol-2-yl)-5-(3-carboxymethoxyphenyl)-2-(4-sulfophenyl)-2H-tetrazolium). Results obtained with the p38 MAPK inhibitor are in agreement with published results using a different p38α MAPK inhibitor.

### Example 2

### p38α MAPK Inhibition Reduces MM Cell Growth and Proliferation

Phosphorylation of p38α MAPK in MM cells was substantially suppressed by Compound 57, while phosphorylation of p38α MAPK in BMSC was partially suppressed. Neither p38α MAPK inhibition nor high concentrations of Compound 57 (tested up to 50-fold excess of active concentration) affected BMSC viability.

A 72-hour exposure to p38 MAPK inhibitor Compound 57 causes a reduction in proliferation of about 20% in each of four MM cell lines tested (U266B, RPMI, ARH-77, and IM9) (FIG. 5).

### Example 3

### p38α MAPK Inhibition Blocks Cytokine Secretion by Multiple Myeloma and Bone Marrow Stromal Cells

The BM microenvironment contributes to MM cell drug resistance and to osteolytic lesions in adjacent bone. The consequences to patients include tumor growth and disease progression, and bone fractures and pain. Cytokines produced by MM cells, or marrow cells in response to MM cells include TNFα, IL-1β, IL-6, VEGF, and PGE2. Primary among these is IL-6, which promotes MM growth and survival, and resistance to chemotherapy.

In addition to supporting the primary tumor, a number of the cytokines produced by MM and marrow cells promote development and activation of osteoclasts. These cells display an inappropriately high level of activity in MM and are responsible for the development of osteolytic lesions. Factors thought to play a primary role in this aspect of MM include IL-11, MIP-1 and RANKL.

p38 MAPK controls secretion of these factors in-vitro in systems representing the BM milieu. To demonstrate this with p38 MAPK inhibitor Compound 57 of Table 2, various models were used including MM cells cultured alone, primary human BMSC cultured alone, MM cells co-cultured with BMSC, and BMSC cultured in the presence of cytokines which are elevated in MM.

TNF-α

Although circulating levels of TNFα are elevated in MM patients, and TNFα is present in MM marrow, secretion of this major inflammatory cytokine is not detected in BMSC or MM cultures under basal conditions or using the stimulus set that induced other MM-relevant signaling molecules. Macrophages are major producers of TNFα and are likely to be a primary source of TNFα in MM.

TNFα plays a role in controlling the production of numerous MM-relevant cytokines in marrow systems and is elevated in MM serum and marrow samples. Exposure of whole blood to Compound 57 caused dose-dependent reduction in LPS-induced TNF-α secretion (FIG. 6).

With regard to inflammatory prostanoids, p38 MAPK inhibitor Compound 57 inhibited LPS-induced COX-2 mRNA. PGE2 levels were also reduced in LPS-induced human whole blood treated with p38 MAPK inhibitor Compound 57, consistent with reduced COX-2 protein and mRNA levels. By virtue of its impact on COX-2 protein synthesis, the p38 MAPK inhibitor Compound 57 blocked PGE2 synthesis but did not directly inhibit the activity of the purified COX-2 enzyme, nor did it inhibit the activity of a platelet-derived preparation of COX-1. The results with p38 MAPK inhibitor Compound 57 are consistent with a literature report demonstrating that p38 MAP kinase mediates COX-2 mRNA induction.

Interleukin-1β

IL-1β illustrates the importance of amplification loops in myeloma-induced cytokine stimulation. IL-1β plays a well-documented role in the induction of multiple cytokines in a “cytokine network” of feedback loops, including strong induction of IL-6. The ability of IL-1 to increase mRNA expression and induce secretion of the MM-relevant cytokines from MM/BMSC cultures is evidence of this role. Exposure of human whole blood (undiluted) to Compound 57 caused a dose-dependent reduction in LPS-induced IL-1β release (FIG. 7).

Interleukin-6

p38% MAPK inhibition potently blocked IL-6 production and secretion MM/BMSC cell co-cultures (FIG. 8), and by MM and BMSC cells. The observed reduction resulted, at least in part, from inhibition of IL-6 mRNA production (data not shown).

Interleukin-11 (IL-11)

IL-11 elicits pro-fibrotic responses and promotes osteoclast-driven bone resorption. In both BMSC and MM/BMSC cultures, 100 nM concentrations of Compound 57 blocked IL-1 and TGFβ powerfully induction of IL-11 at the mRNA and protein levels (FIG. 9).

Vascular Endothelial Growth Factor (VEGF)

In addition to promoting vascularization, VEGF is a growth-supporting factor for MM cells. Neovascularization of MM tumors in the marrow allows growth beyond diffusion limitations for gas and nutrient exchange. VEGF is secreted from MM contact-activated BMSC, and this secretion is inhibited by p38 MAPK inhibitor Compound 4 (FIG. 10).

### Example 4

### Receptor Activator of Nuclear Factor-κB Ligand (RANKL)

RANKL is a powerful inducer of osteoclast differentiation and activation. This transcription factor is highly activated in MM cells and is activated by p38α MAPK. For this reason, experimental therapeutic strategies for MM bone lesions and other osteolytic diseases are focusing on inhibition of RANK signaling, including activation of NFκB. This transcription factor is highly activated in MM cells, and is activated by p38 MAPK. NFκB inhibition is believed to be a major mechanism in several demonstrated therapies for MM.

RANKL mRNA is powerfully induced by TGFβ and IL-1 in BMSC and MM/BMSC cultures. Exposure to 100 nM p38 MAPK inhibitors Compound 57 or the hydrochloride salt thereof blocked this induction, as well as basal expression of RANKL mRNA (FIG. 11). In addition, 100 nM of these p38 MAPK inhibitors blocks the weaker induction of RANKL by IL-6 in BMSC.

### Example 5

### Macrophage Inflammatory Protein-1 Alpha (MIP-1α)

MM models show that MIP-la is necessary for the development of osteolytic lesions. MIP-1α elevation in MM patient serum correlates with disease severity. Unlike many MM-relevant cytokines present in the marrow microenvironment, which are produced mainly by the activated BMSC, MM cells are the primary source of MIP-1α. IL-1 further induces MIP-1α production. The p38 MAPK inhibitors of Table 2, for example Compound 57 substantially reduce both IL-1-induced and untreated production of MIP-1α in MM/BMSC co-cultures (FIG. 12).

### Example 6

### Anti-Osteoclastic Effects of p38α MAPK Inhibition In Vitro

Osteoclast differentiation and activation is promoted by marrow and circulating factors that include MIP-1α, RANKL, and macrophage colony stimulating factor (M-CSF). Activated osteoclasts release osteolytic enzymes and protons, which cause bone degradation followed by resorption. This process can be observed in MM patients by measuring increased serum markers of bone resorption including elevated calcium that can reach nephrotoxic levels.

Inhibition of p38α MAPK prevented osteoclast differentiation in two types of standard in vitro models. In the first, primary murine marrow derived osteoclast precursors are cultured in the presence of di-hydroxy vitamin D to induce differentiation. Mature osteoclast formation is prevented by the addition of the p38 MAPK inhibitors (Compounds 57 and 162) in a dose dependent manner, with complete blockage at 100 nM of these exemplary inhibitors (FIG. 13). In the second model, primary human marrow-derived osteoclast precursors are cultured in the presence of dihydroxy vitamin D [(OH)₂D3] to induce differentiation. In both cases, 100 nM of the exemplary p38 MAPK inhibitors prevents osteoclast formation as measured by tartrate resistant acid phosphatase (TRAP) staining or by staining with 23c6 (a mature osteoclast cell surface marker)(FIG. 14).

### Example 7

### Anti-Cytokine Effects of p38 MAPK Inhibitors In-Vivo: Bone Erosion and Related Features of Chronic Arthritis

Chronic arthritis shares certain disease mechanisms, as well as signs and symptoms, with the bone disease observed in 80% of MM patients. Specifically, osteolytic lesions are present in both and are characterized by osteoclast differentiation and activation resulting in bone erosion, fractures and pain.

Rats treated intradermally with chick type II collagen develop arthritic lesions in the hind paws similar to human arthritic joints with respect to accumulation of pannus tissue, presence of inflammatory cells, and increased expression of inflammatory cytokines. Therefore, this model was used to evaluate the therapeutic potential of p38 MAPK inhibitors Compound 57 in rats with established disease. Animals diagnosed with arthritis on day 10 were enrolled into the study and treated orally once a day with vehicle (0.003% HCl in water), 10 mg/kg or 40 mg/kg of Compound 57. Animals were scored daily by an observer blinded with respect to treatment regimen.

At the completion of the study on Day 28, radiographic images were analyzed in a blinded manner, based upon the extent of soft tissue swelling, joint space narrowing, and bone destruction. Animal weights at the beginning and end of the study were also recorded, as weight loss is associated with disease progression in this model. Compared to vehicle-treated animals, those treated with 40 mg/kg of any of Compound 57 showed a statistically significant improvement in clinical score from the third day of treatment to the completion of the study, with little progression of disease noted. The effect of a 10 mg/kg dose was intermediate between the vehicle and 40 mg/kg groups (FIG. 15).

### Example 8

### A p38-α MAP Kinase Inhibitor Inhibits Human Myeloma Cell Growth In Vivo

The p38 MAPK signaling pathway plays an important role in different pathological conditions and specific inhibitors of p38 alpha and beta MAPK block production of the major inflammatory cytokines. Human multiple myeloma (MM) is an incurable neoplasm of terminally differentiated B cells and MM disease progression is affected by immunoregulatory elements such as IL-6 and other cytokines. Recent literature indicates that an important function of p38 MAP kinase is the generation of signals of critical value to the control of normal and malignant hematopoiesis by cytokines and growth factors. The effect of p38 alpha MAP kinase specific inhibitor Compound 57, on human myeloma (RPM18266) tumor growth in an immunodeficient beige-nude-xid mouse xenograft plasmacytoma model. Treatment (90 mg/kg/bid by oral gavage) is initiated in mice with palpable tumor size (˜200 mm³) as judged by tumor volume. In mice with palpable tumors, 21 days of Compound 57 treatment significantly reduced tumor volume. When Compound 57 treatment is initiated in mice with pronounced tumor size (˜800 mm³), there remains a significant reduction in tumor volume. Histological assessment at the end of dosing from animals administered the exemplary compounds or vehicle demonstrates a significant reduction in tumor volume and number of neoformed microvessels in the p38 MAPK Compound 57 treatment groups. These inhibitors also significantly reduce HSP27 and p38 expressions in tumor cells.

In conclusion, p38 inhibitors inhibit human myeloma cell growth in vivo via p38 MAP kinase signaling pathway. The current study provides strong in vivo evidence supporting use of p38 inhibitor therapy in patients with multiple myeloma.

### Example 9

### p38 MAPK Inhibition to Treat a Subject with Osteolytic Lesions Associated with Multiple Myeloma

A patient is diagnosed with multiple myeloma and presents with a number of holes or osteolytic lesions. The osteolytic lesions are caused by the rapid growth of myeloma cells, which displace osteoblasts, and disrupting the balance of bone creation and destruction. A therapeutic amount of p38 MAPK inhibitor Compound 57 as a hydrochloric salt is administered. Osteoblast activity is increased relative to osteoclast activity. Pain from bone destruction, fracture and the like is reduced.

### Example 10

### MAPK Inhibition in Combination with a Hypercalcium Chemotherapeutic for Treating a Subject with Osteolytic Lesions Associated with Multiple Myeloma

A patient is diagnosed with multiple myeloma and presents with a number of holes or osteolytic lesions. The osteolytic lesions are caused by the rapid growth of myeloma cells, which displace osteoblasts, and disrupting the balance of bone creation and destruction. A therapeutic amount of p38 MAPK inhibitor Compound 57 in a hydrochloric salt form is administered. Osteoblast activity is increased relative to osteoclast activity. Pain from bone destruction, fracture and the like is reduced.

What is claimed is:

1. A method of preventing or reducing an osteolytic lesion associated with multiple myeloma (MM), comprising:
identifying a subject suffering from the osteolytic lesion; and
providing the subject with an effective amount of a p38 MAP kinase inhibitor such that the osteolytic lesion is reduced or eliminated.

2. The method of claim 1, wherein the p38 inhibitor is of the formula: 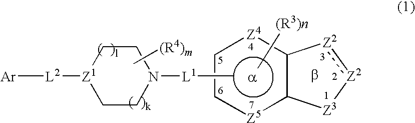
and the pharmaceutically acceptable salts thereof, or a pharmaceutical composition thereof, wherein [symbol]
represents a single or double bond;
one Z² is CA or CR⁶A and the other is CR¹ or CR¹₂, wherein each R¹ is independently hydrogen is alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, R₃Si, and NO₂, wherein each R is independently H, alkyl, alkenyl or aryl or the heteroforms thereof;
R⁶ is H, alkyl, alkenyl, alkynyl, aryl, arylalkyl, acyl, aroyl, heteroaryl, heteroalkyl, heteroalkenyl, heteroalkynyl, heteroalkylaryl, SOR, SO₂R, RCO, COOR, alkyl-COR, SO₃R, CONR₂, SO₂NR₂, CN, CF₃, or R₃Si wherein each R is independently H, alkyl, alkenyl or aryl or the heteroforms thereof;
A is —Wᵢ—COXⱼY wherein Y is COR² wherein R² is hydrogen, straight or branched chain alkyl, alkenyl, alkynyl, aryl, arylalkyl, heteroalkyl, heteroaryl, or heteroarylalkyl, each optionally substituted with halo, alkyl, SR, OR, NR₂, OCOR, NRCOR, NRCONR₂, NRSO₂R, NRSO₂NR₂, OCONR₂, CN, COOR, CONR₂, COR, or R₃Si wherein each R is independently H, alkyl, alkenyl or aryl or the heteroforms thereof; or
wherein R² is OR, NR₂, NRCONR₂, OCONR₂, NRSO₂NR₂, heteroarylalkyl, COOR, NRNR₂, heteroaryl, heteroaryloxy, heteroaryl-NR, or NROR wherein each R is independently H, alkyl, alkenyl or aryl or the heteroforms thereof, and wherein two R attached to the same N atom may form a 3-8 member ring and wherein said ring may further be substituted by alkyl, alkenyl, alkynyl, aryl, arylalkyl, heteroaryl, heteroalkyl, heteroarylalkyl, each optionally substituted with halo, SR, OR, NR₂, OCOR, NRCOR, NRCONR₂, NRSO₂R, NRSO₂NR₂, OCONR₂, or R₃Si wherein each R is independently H, alkyl, alkenyl or aryl or the heteroforms thereof wherein two R attached to the same atom may form a 3-8 member ring, optionally substituted as above defined, and
each of W and X is substituted or unsubstituted alkylene or alkenylene, each of 2-6A, or
Y is tetrazole; 1,2,3-triazole; 1,2,4-triazole; or imidazole;
each of i and j is independently 0 or 1;
Z³ is NR⁷ or O;
R⁷ is H or is optionally substituted alkyl, alkenyl, alkynyl, aryl, arylalkyl, acyl, aroyl, heteroaryl, heteroalkyl, heteroalkenyl, heteroalkynyl, or heteroalkylaryl, or is SOR, SO₂R, RCO, COOR, alkyl-COR, SO₃R, CONR₂, SO₂NR₂, CN, CF₃, NR₂, OR, alkyl-SR, alkyl-SOR, alkyl-SO₂R, alkyl-OCOR, alkyl-COOR, alkyl-CN, alkyl-CONR₂, or R₃Si, wherein each R is independently H, alkyl, alkenyl or aryl or heteroforms thereof;
each R³ is independently halo, alkyl, heteroalkyl, OCOR, OR, NRCOR, SR, or NR₂, wherein R is H, alkyl or aryl or the heteroforms thereof;
n is 0-3;
L¹ is CO, SO₂ or alkylene (1-4C);
L² is alkylene (1-4C) or alkenylene (2-4C) optionally substituted with one or two moieties selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, and R₃Si, wherein each R is independently H, alkyl, alkenyl or aryl or the heteroforms thereof, and wherein two substituents on L² can be joined to form a non-aromatic saturated or unsaturated ring that includes 0-3 heteroatoms which are O, S and/or N and which contains 3 to 8 members or said two substituents can be joined to form a carbonyl moiety or an oxime, oximeether, oximeester or ketal of said carbonyl moiety;
each R⁴ is independently selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, R₃Si, and NO₂, wherein each R is independently H, alkyl, alkenyl or aryl or the heteroforms thereof, and two of R⁴ on adjacent positions can be joined to form a fused, optionally substituted aromatic or nonaromatic, saturated or unsaturated ring which contains 3-8 members, or R⁴ is ═O or an oxime, oximeether, oximeester or ketal thereof;
m is 0-4;
Z¹ is CR⁵ or N wherein R⁵ is hydrogen or OR, NR₂, SR or halo, wherein each R is independently H, alkyl, alkenyl or aryl or the heteroforms thereof;
each of l and k is an integer from 0-2 wherein the sum of l and k is 0-3;
Ar is an aryl group substituted with 0-5 noninterfering substituents selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, R₃Si, and NO₂, wherein each R is independently H, alkyl, alkenyl or aryl or the heteroforms thereof, and wherein two of said optional substituents on adjacent positions can be joined to form a fused, optionally substituted aromatic or nonaromatic, saturated or unsaturated ring which contains 3-8 members.

3. The method of claim 1, wherein the osteolytic lesion is reduced or eliminated by reducing production of an osteolytic lesion-promoting cytokine.

4. The method of claim 3, wherein the MM-related cytokine is selected from the group consisting of IL-6, VEGF, IL-11, and PGE-2.

5. The method of claim 1, wherein osteolytic lesions are reduced or eliminated by inhibiting osteoclast development and activation.

6. The method of claim 1, wherein a reduction in soft tissue swelling, joint space narrowing, or bone destruction comprise indicia of osteolytic lesion reduction.

7. A method of inhibiting osteolytic lesion-promoting cytokine secretion from multiple myeloma cells, comprising:
providing a p38 MAP kinase inhibitor to a subject suffering from MM associated osteolytic lesions, wherein the secretion rate of a MM-related cytokine is reduced as compared to the secretion rate of a MM-related cytokine of untreated MM cells.

8. The method of claim 7, wherein the MM-related cytokine is selected from the group consisting of IL-6, VEGF, IL-11, and PGE-2.

9. The method of claim 7, wherein the p38 inhibitor is of the formula: 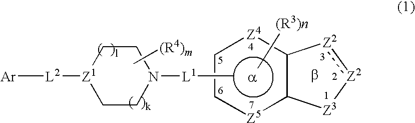
and the pharmaceutically acceptable salts thereof, or a pharmaceutical composition thereof, wherein [symbol]
represents a single or double bond;
one Z² is CA or CR⁶A and the other is CR¹ or CR₁₂, wherein each R¹ is independently hydrogen is alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, R₃Si, and NO₂, wherein each R is independently H, alkyl, alkenyl or aryl or the heteroforms thereof;
R⁶ is H, alkyl, alkenyl, alkynyl, aryl, arylalkyl, acyl, aroyl, heteroaryl, heteroalkyl, heteroalkenyl, heteroalkynyl, heteroalkylaryl, SOR, SO₂R, RCO, COOR, alkyl-COR, SO₃R, CONR₂, SO₂NR₂, CN, CF₃, or R₃Si wherein each R is independently H, alkyl, alkenyl or aryl or the heteroforms thereof;
A is —Wᵢ—COXⱼY wherein Y is COR² wherein R² is hydrogen, straight or branched chain alkyl, alkenyl, alkynyl, aryl, arylalkyl, heteroalkyl, heteroaryl, or heteroarylalkyl, each optionally substituted with halo, alkyl, SR, OR, NR₂, OCOR, NRCOR, NRCONR₂, NRSO₂R, NRSO₂NR₂, OCONR₂, CN, COOR, CONR₂, COR, or R₃Si wherein each R is independently H, alkyl, alkenyl or aryl or the heteroforms thereof; or
wherein R² is OR, NR₂, NRCONR₂, OCONR₂, NRSO₂NR₂, heteroarylalkyl, COOR, NRNR₂, heteroaryl, heteroaryloxy, heteroaryl-NR, or NROR wherein each R is independently H, alkyl, alkenyl or aryl or the heteroforms thereof, and wherein two R attached to the same N atom may form a 3-8 member ring and wherein said ring may further be substituted by alkyl, alkenyl, alkynyl, aryl, arylalkyl, heteroaryl, heteroalkyl, heteroarylalkyl, each optionally substituted with halo, SR, OR, NR₂, OCOR, NRCOR, NRCONR₂, NRSO₂R, NRSO₂NR₂, OCONR₂, or R₃Si wherein each R is independently H, alkyl, alkenyl or aryl or the heteroforms thereof wherein two R attached to the same atom may form a 3-8 member ring, optionally substituted as above defined, and
each of W and X is substituted or unsubstituted alkylene or alkenylene, each of 2-6 Å, or
Y is tetrazole; 1,2,3-triazole; 1,2,4-triazole; or imidazole;
each of i and j is independently 0 or 1;
Z³ is NR⁷ or O;
R⁷ is H or is optionally substituted alkyl, alkenyl, alkynyl, aryl, arylalkyl, acyl, aroyl, heteroaryl, heteroalkyl, heteroalkenyl, heteroalkynyl, or heteroalkylaryl, or is SOR, SO₂R, RCO, COOR, alkyl-COR, SO₃R, CONR₂, SO₂NR₂, CN, CF₃, NR₂, OR, alkyl-SR, alkyl-SOR, alkyl-SO₂R, alkyl-OCOR, alkyl-COOR, alkyl-CN, alkyl-CONR₂, or R₃Si, wherein each R is independently H, alkyl, alkenyl or aryl or heteroforms thereof;
each R³ is independently halo, alkyl, heteroalkyl, OCOR, OR, NRCOR, SR, or NR₂, wherein R is H, alkyl or aryl or the heteroforms thereof;
n is 0-3;
L¹ is CO, SO₂ or alkylene (1-4C);
L² is alkylene (1-4C) or alkenylene (2-4C) optionally substituted with one or two moieties selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, and R₃Si, wherein each R is independently H, alkyl, alkenyl or aryl or the heteroforms thereof, and wherein two substituents on L² can be joined to form a non-aromatic saturated or unsaturated ring that includes 0-3 heteroatoms which are O, S and/or N and which contains 3 to 8 members or said two substituents can be joined to form a carbonyl moiety or an oxime, oximeether, oximeester or ketal of said carbonyl moiety;
each R⁴ is independently selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, R₃Si, and NO₂, wherein each R is independently H, alkyl, alkenyl or aryl or the heteroforms thereof, and two of R⁴ on adjacent positions can be joined to form a fused, optionally substituted aromatic or nonaromatic, saturated or unsaturated ring which contains 3-8 members, or R⁴ is ═O or an oxime, oximeether, oximeester or ketal thereof;
m is 0-4;
Z¹ is CR⁵ or N wherein R⁵ is hydrogen or OR, NR₂, SR or halo, wherein each R is independently H, alkyl, alkenyl or aryl or the heteroforms thereof;
each of l and k is an integer from 0-2 wherein the sum of l and k is 0-3;
Ar is an aryl group substituted with 0-5 noninterfering substituents selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, R₃Si, and NO₂, wherein each R is independently H, alkyl, alkenyl or aryl or the heteroforms thereof, and wherein two of said optional substituents on adjacent positions can be joined to form a fused, optionally substituted aromatic or nonaromatic, saturated or unsaturated ring which contains 3-8 members.

10. A method of inhibiting osteoclast development and activation comprising:
identifying an individual with MM mediated osteoclast activation; and
providing a p38 MAP kinase inhibitor to the individual, such that MM mediated osteoclast activation is reduced.

11. The method of claim 10, wherein the p38 inhibitor is of the formula: 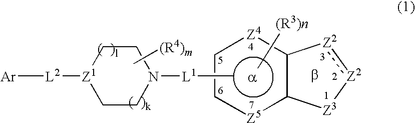
and the pharmaceutically acceptable salts thereof, or a pharmaceutical composition thereof, wherein [symbol]
represents a single or double bond;
one Z² is CA or CR⁶A and the other is CR¹ or CR₁₂, wherein each R¹ is independently hydrogen is alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, R₃Si, and NO₂, wherein each R is independently H, alkyl, alkenyl or aryl or the heteroforms thereof;
R⁶ is H, alkyl, alkenyl, alkynyl, aryl, arylalkyl, acyl, aroyl, heteroaryl, heteroalkyl, heteroalkenyl, heteroalkynyl, heteroalkylaryl, SOR, SO₂R, RCO, COOR, alkyl-COR, SO₃R, CONR₂, SO₂NR₂, CN, CF₃, or R₃Si wherein each R is independently H, alkyl, alkenyl or aryl or the heteroforms thereof;
A is —Wᵢ—COXⱼY wherein Y is COR² wherein R² is hydrogen, straight or branched chain alkyl, alkenyl, alkynyl, aryl, arylalkyl, heteroalkyl, heteroaryl, or heteroarylalkyl, each optionally substituted with halo, alkyl, SR, OR, NR₂, OCOR, NRCOR, NRCONR₂, NRSO₂R, NRSO₂NR₂, OCONR₂, CN, COOR, CONR₂, COR, or R₃Si wherein each R is independently H, alkyl, alkenyl or aryl or the heteroforms thereof; or
wherein R² is OR, NR₂, NRCONR₂, OCONR₂, NRSO₂NR₂, heteroarylalkyl, COOR, NRNR₂, heteroaryl, heteroaryloxy, heteroaryl-NR, or NROR wherein each R is independently H, alkyl, alkenyl or aryl or the heteroforms thereof, and wherein two R attached to the same N atom may form a 3-8 member ring and wherein said ring may further be substituted by alkyl, alkenyl, alkynyl, aryl, arylalkyl, heteroaryl, heteroalkyl, heteroarylalkyl, each optionally substituted with halo, SR, OR, NR₂, OCOR, NRCOR, NRCONR₂, NRSO₂R, NRSO₂NR₂, OCONR₂, or R₃Si wherein each R is independently H, alkyl, alkenyl or aryl or the heteroforms thereof wherein two R attached to the same atom may form a 3-8 member ring, optionally substituted as above defined, and
each of W and X is substituted or unsubstituted alkylene or alkenylene, each of 2-6 Å, or
Y is tetrazole; 1,2,3-triazole; 1,2,4-triazole; or imidazole;
each of i and j is independently 0 or 1;
Z³ is NR⁷ or O;
R⁷ is H or is optionally substituted alkyl, alkenyl, alkynyl, aryl, arylalkyl, acyl, aroyl, heteroaryl, heteroalkyl, heteroalkenyl, heteroalkynyl, or heteroalkylaryl, or is SOR, SO₂R, RCO, COOR, alkyl-COR, SO₃R, CONR₂, SO₂NR₂, CN, CF₃, NR₂, OR, alkyl-SR, alkyl-SOR, alkyl-SO₂R, alkyl-OCOR, alkyl-COOR, alkyl-CN, alkyl-CONR₂, or R₃Si, wherein each R is independently H, alkyl, alkenyl or aryl or heteroforms thereof;
each R³ is independently halo, alkyl, heteroalkyl, OCOR, OR, NRCOR, SR, or NR₂, wherein R is H, alkyl or aryl or the heteroforms thereof;
n is 0-3;
L¹ is CO, SO₂ or alkylene (1-4C);
L² is alkylene (1-4C) or alkenylene (2-4C) optionally substituted with one or two moieties selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, and R₃Si, wherein each R is independently H, alkyl, alkenyl or aryl or the heteroforms thereof, and wherein two substituents on L² can be joined to form a non-aromatic saturated or unsaturated ring that includes 0-3 heteroatoms which are O, S and/or N and which contains 3 to 8 members or said two substituents can be joined to form a carbonyl moiety or an oxime, oximeether, oximeester or ketal of said carbonyl moiety;
each R⁴ is independently selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, R₃Si, and NO₂, wherein each R is independently H, alkyl, alkenyl or aryl or the heteroforms thereof, and two of R⁴ on adjacent positions can be joined to form a fused, optionally substituted aromatic or nonaromatic, saturated or unsaturated ring which contains 3-8 members, or R⁴ is ═O or an oxime, oximeether, oximeester or ketal thereof;
m is 0-4;
Z¹ is CR⁵ or N wherein R⁵ is hydrogen or OR, NR₂, SR or halo, wherein each R is independently H, alkyl, alkenyl or aryl or the heteroforms thereof;
each of l and k is an integer from 0-2 wherein the sum of l and k is 0-3;
Ar is an aryl group substituted with 0-5 noninterfering substituents selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, R₃Si, and NO₂, wherein each R is independently H, alkyl, alkenyl or aryl or the heteroforms thereof, and wherein two of said optional substituents on adjacent positions can be joined to form a fused, optionally substituted aromatic or nonaromatic, saturated or unsaturated ring which contains 3-8 members.
